(19) United States
(12) Patent Application Publication
(10) Pub. No.: US 2015/0223782 A1
(43) Pub. Date: Aug. 13, 2015

(54) ULTRASOUND DIAGNOSIS APPARATUS

(71) Applicants: Kabushiki Kaisha Toshiba, Minato-ku (JP); Toshiba Medical Systems Corporation, Otawara-shi (JP)

(72) Inventors: Hitoshi YAMAGATA, Otawara (JP); Yasuhiko ABE, Otawara (JP)

(73) Assignees: Kabushiki Kaisha Toshiba, Minato-ku (JP); Toshiba Medical Systems Corporation, Otawara-shi (JP)

(21) Appl. No.: 14/678,469

(22) Filed: Apr. 3, 2015

Related U.S. Application Data
(63) Continuation of application No. PCT/JP2013/077178, filed on Oct. 4, 2013.

(30) Foreign Application Priority Data
Oct. 4, 2012 (JP) 2012-222591

(51) Int. Cl. A61B 8/00; A61B 5/08; A61B 7/04; A61B 8/12; A61B 5/0402

(52) U.S. Cl. CPC A61B 8/543; A61B 8/12; A61B 5/0402; A61B 7/04; A61B 5/0816; A61B 8/4444

(57) ABSTRACT

An ultrasound diagnosis apparatus includes an ultrasound transducer and a controller. The ultrasound transducer transmits and receives ultrasound waves while being inserted in a subject to acquire biological information of a predetermined site of the subject. The controller controls the ultrasound transducer to transmit ultrasound waves based on a trigger signal that is set according to conditions of the predetermined site that exhibits periodic motion, or that is obtained according to the conditions.

### CROSS-REFERENCE TO RELATED APPLICATION

This application is based upon and claims the benefit of priority from Japanese Patent Application No. 2012-222591, filed Apr. 10, 2012; the entire contents of which are incorporated herein by reference.

### FIELD

Embodiments described herein relate generally to an ultrasound diagnosis apparatus.

### BACKGROUND

A medical image diagnosis apparatus is a device that creates, for examination and diagnosis, a medical image (tomographic image, blood flow image, etc.) from information on tissues in a subject without surgical removal of the tissues. Examples of such medical image diagnosis apparatus include X-ray diagnosis apparatuses, X-ray CT (Computed Tomography) apparatuses, MRI (Magnetic Resonance Imaging) apparatuses, and ultrasound diagnosis apparatuses.

In one example, after the image of a subject is captured, the medical image is stored in a medical image archive system (e.g., PACS; Picture Archiving and Communication Systems) in a healthcare institution. After that, a radiologist retrieves the medical image from the medical image archive system to interpret it. In another example, after the image of a subject is captured, the medical image is displayed immediately (in real time) for inspection by a doctor or the like. That is, medical images may be used so that a doctor or the like can know about conditions inside the subject's body at the point. In still another example, medical images may be used to monitor conditions inside the subject's body during a specific period for a follow-up. An ultrasound diagnosis apparatus may be used for this monitoring. In other words, there may be cases where the ultrasound diagnosis apparatus is used in consideration of a point that it does not cause radiation exposure to the subject.

When conditions inside the subject's body are monitored for a certain period, it may be difficult to keep the subject in a gantry (an X-ray CT apparatus, an MRI apparatus, etc.) depending on the length of the period. The same is applied to X-ray diagnosis apparatuses that require the subject to be kept between an X-ray irradiator and a detector. In contrast, the ultrasound diagnosis apparatus does not need a gantry or the like. The ultrasound diagnosis apparatus transmits/receives ultrasound waves to/from an observation site with an ultrasound probe or the like, thereby obtaining information on body tissues to be imaged. In addition, the ultrasound diagnosis apparatus does not make noise due to vibration of a gradient coil unlike MRI apparatuses.

However, if the ultrasound probe obtains ultrasound images of the body tissues from outside the body, there may be the influence of tissues (bones, lungs, etc.) present in the way to the desired observation site from the body surface. To solve the problem, the ultrasound diagnosis apparatus is provided with a transesophageal echocardiography (TEE) probe. The TEE probe transmits/receives ultrasound waves in the esophagus or the upper digestive tract, and thereby can obtain the ultrasound image of a desired observation site without the influence of the tissues as above mentioned.

As an example of the structure, the TEE probe includes a guiding hollow tube part having a predetermined length, an end part having an ultrasound transducer, and a curved part that connects the guiding hollow tube part with the end part. The portion from the guiding hollow tube part to the end part is inserted in the body cavity, for example, in the upper digestive tract, such as the esophagus and the stomach. Therefore, the guiding hollow tube part is formed to be flexible. The end of the guiding hollow tube part opposite to the end part is connected to a gripper. The gripper is held by the operator, and provided with an operation unit used to manipulate the curved part and the end part. A wire is strung from the gripper through the guiding hollow tube part to the end part. The wire is used to bend the curved part.

By the operation on the gripper, the wire is driven, and the curved part is bent. Thus, the end part is pointed in a predetermined direction. While the end part is pointed in the predetermined direction, the ultrasound transducer of the end part transmits/receives ultrasound waves to/from a desired observation site. With this, the ultrasound diagnosis apparatus can obtain an image that indicates, for example, conditions of the heart from a predetermined location in the esophagus.

In the ultrasound diagnosis apparatus, the ultrasound transducer transmits/receives ultrasound waves, and this may cause a temperature rise. For example, piezoelectric elements generate heat due to internal loss caused by the conversion of voltage applied thereto to ultrasound waves. When conditions inside the subject's body are monitored for a predetermined period, the temperature rise may interfere the continuation of the monitoring. As described above, the part that transmits/receives ultrasound waves may be inserted in the subject's body, and therefore, there is a need to suppress the temperature rise.

### BRIEF DESCRIPTION OF THE DRAWINGS

FIG. 1 is a schematic perspective view of an ultrasound diagnosis apparatus;

FIG. 2A is a schematic side view of an end part;

FIG. 2B provides schematic cross sections taken along lines A-A′ and B-B′ in FIG. 2A, illustrating the positional relationship of parts therein;

FIG. 2C is a schematic cross section of an ultrasound transducer illustrated in FIG. 2A, to which offset is applied;

FIG. 2D is a schematic perspective view of a flexible printed circuit board;

FIG. 3A is a schematic perspective view of the ultrasound transducer;

FIG. 3B is a schematic perspective view of the ultrasound transducer;

FIG. 3C is a schematic perspective view of the ultrasound transducer;

FIG. 3D is a schematic perspective view of the ultrasound transducer;

FIG. 4 is a schematic block diagram illustrating an example of the functional structure of an end part of an ultrasound diagnosis apparatus according to a first embodiment;

FIG. 5 is a schematic block diagram illustrating an example of the functional structure of a main body of the ultrasound diagnosis apparatus of the first embodiment;

FIG. 6 is a schematic diagram of an example of a B-mode image generated by a generating unit of the first embodiment;

FIG. 7A is a schematic diagram of an example of a Doppler spectrum image generated by the generating unit of the first embodiment;

FIG. 7B is a schematic diagram of an example of ECG (electrocardiogram) waveform and the Doppler spectrum image generated by the generating unit of the first embodiment;

FIG. 8 is a schematic diagram illustrating a positional relationship for obtaining the B-mode image illustrated in FIG. 6;

FIG. 9 is a flowchart schematically illustrating the operation of the ultrasound diagnosis apparatus of the first embodiment;

FIG. 10 is a flowchart schematically illustrating the operation of an ultrasound diagnosis apparatus according to a third embodiment;

FIG. 11 is a schematic block diagram illustrating an example of the functional structure of a main body of an ultrasound diagnosis apparatus according to a fifth embodiment;

FIG. 12 is a flowchart schematically illustrating the operation of the ultrasound diagnosis apparatus of the fifth embodiment;

FIG. 13 is a flowchart schematically illustrating the operation of the ultrasound diagnosis apparatus of the fifth embodiment; and

FIG. 14 is a flowchart schematically illustrating the operation of the ultrasound diagnosis apparatus of the fifth embodiment.

### DETAILED DESCRIPTION

In general, according to one embodiment, an ultrasound diagnosis apparatus includes an ultrasound transducer and a controller. The ultrasound transducer transmits and receives ultrasound waves while being inserted in a subject to acquire biological information of a predetermined site of the subject. The controller controls the ultrasound transducer to transmit ultrasound waves based on a trigger signal that is set according to conditions of the predetermined site that exhibits periodic motion, or that is obtained according to the conditions.

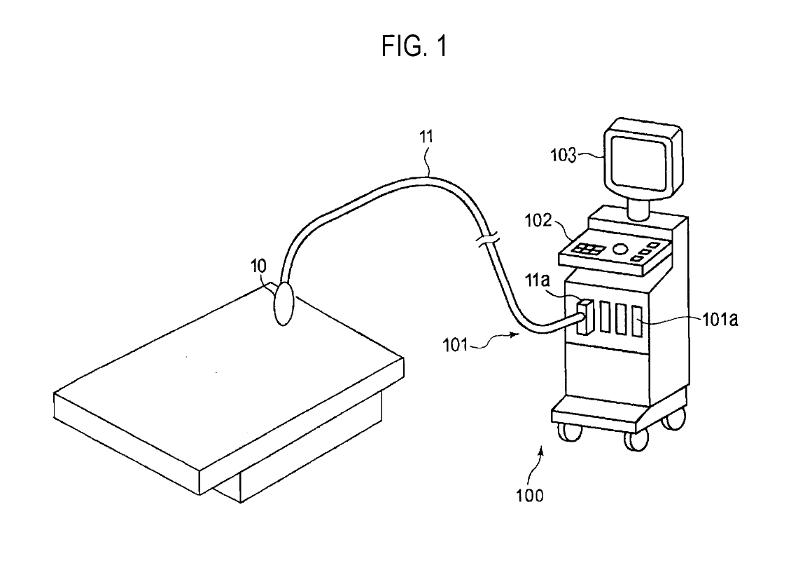

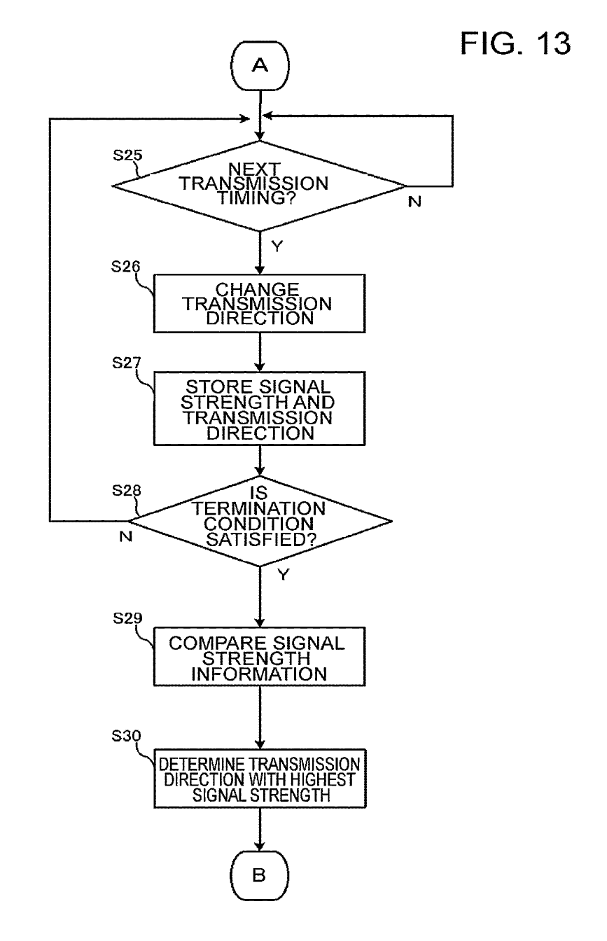

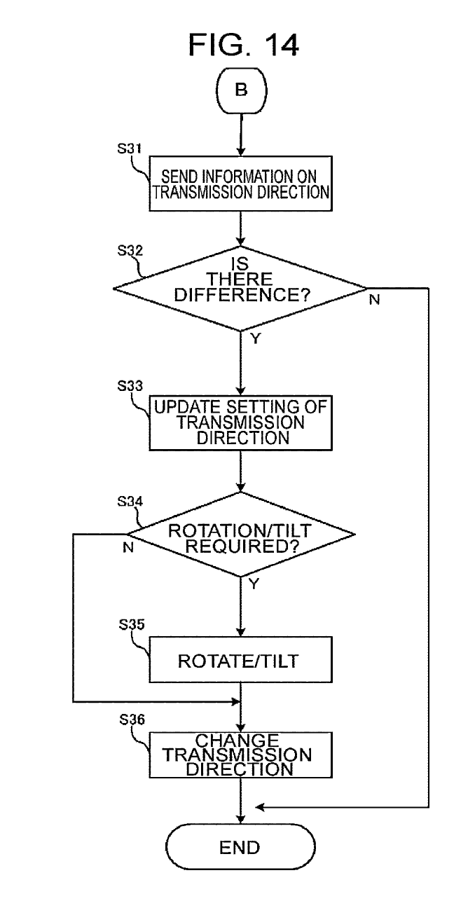

Referring to FIGS. 1 to 14, a description is given of an ultrasound diagnosis apparatus according to first to sixth embodiments.

### First Embodiment

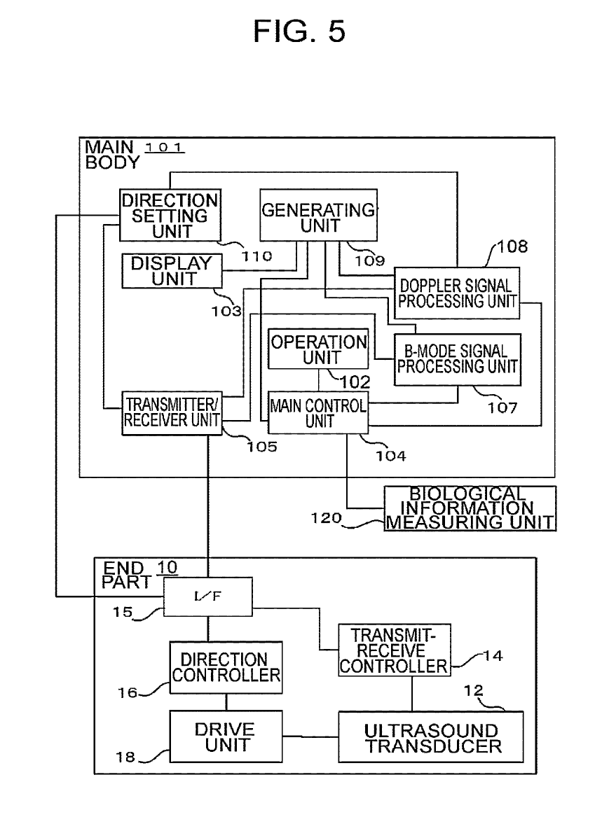

The overview of the overall structure of the ultrasound diagnosis apparatus 100 according to the first embodiment is described first with reference to FIG. 1. FIG. 1 is an external view of the ultrasound diagnosis apparatus 100 for explaining the overview of its structure. The ultrasound diagnosis apparatus 100 of the first embodiment receives an analysis result according to the setting of biological information (ECG (electrocardiogram) waveform, etc.) from a biological information measuring unit 120 (see FIG. 5), and intermittently captures images.

As illustrated in FIG. 1, the ultrasound diagnosis apparatus 100 of the embodiment includes a main body 101, an end part 10 and the like. The end part 10 and the main body 101 are connected through a cable 11. In the example of FIG. 1, a connector 11a is provided to the end of the cable 11 for connection to the main body 101. The main body 101 is provided with connection parts 101a that are connectable to the connector 11a. The main body 101 includes an operation unit 102 that is used to operate the ultrasound diagnosis apparatus 100 and a display unit 103 that displays an image generated by the ultrasound diagnosis apparatus 100 and other images. Incidentally, the illustration of the ultrasound diagnosis apparatus 100 in FIG. 1 is by way of example only. The structure of the main body 101, the arrangement and the structure of the cable 11, the operation unit 102 and the display unit 103, and the like are not limited to those in FIG. 1, and susceptible to various modifications as appropriate. For example, instead of being configured as illustrated in FIG. 1, the main body 101 may be configured as a portable ultrasound diagnosis apparatus.

### <Structure of End Part>

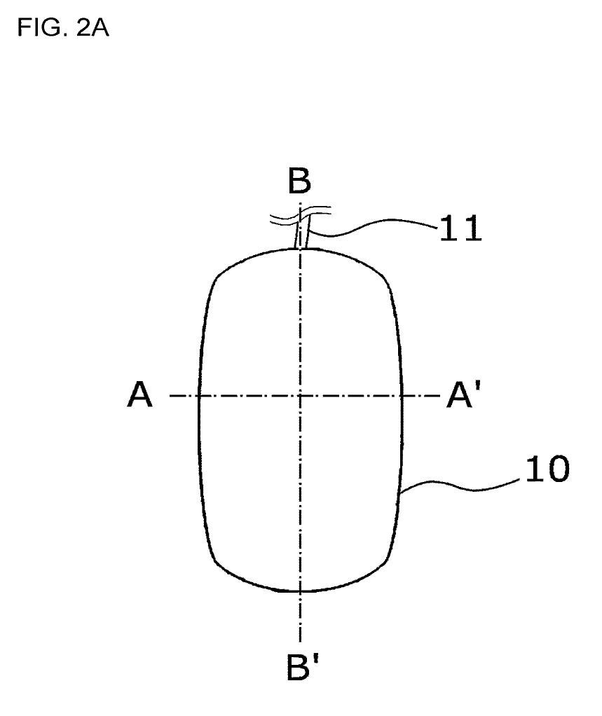

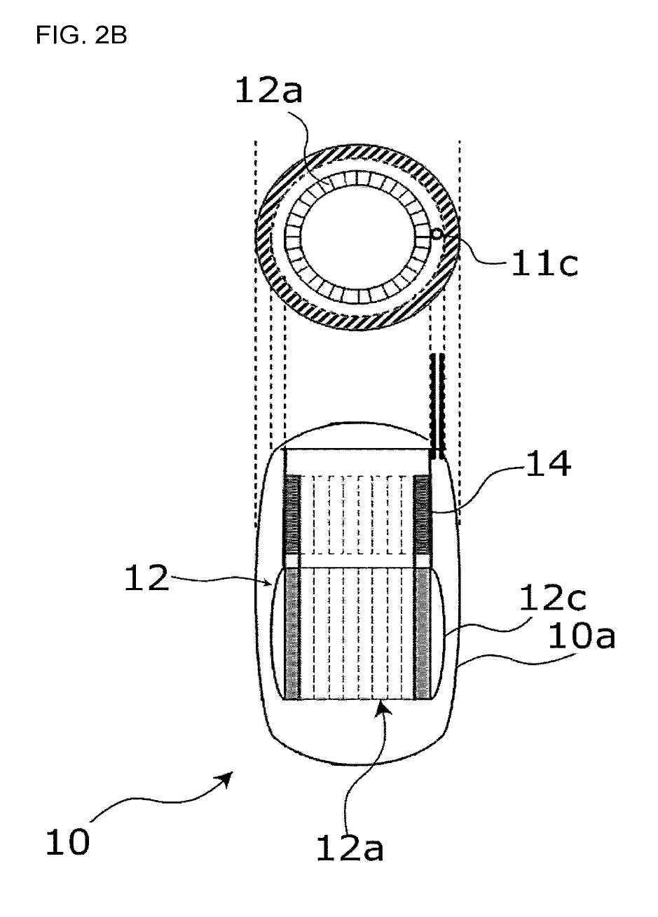

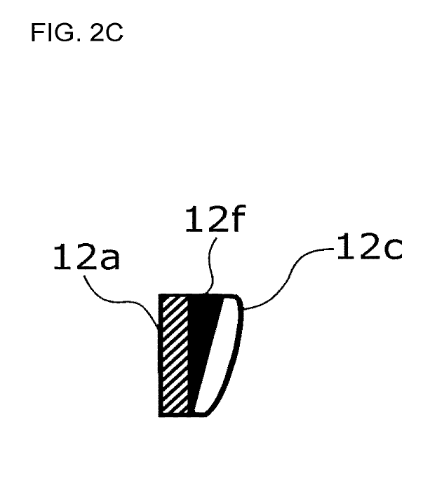

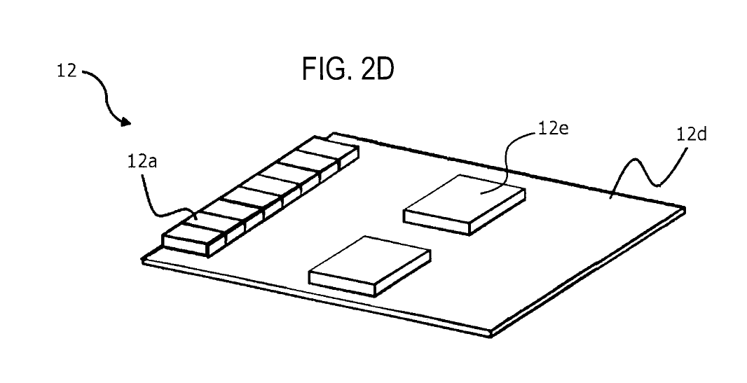

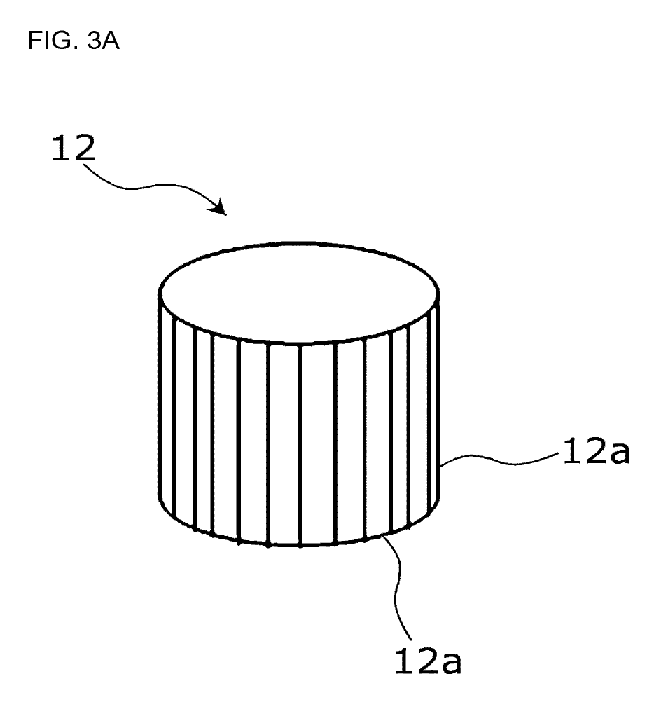

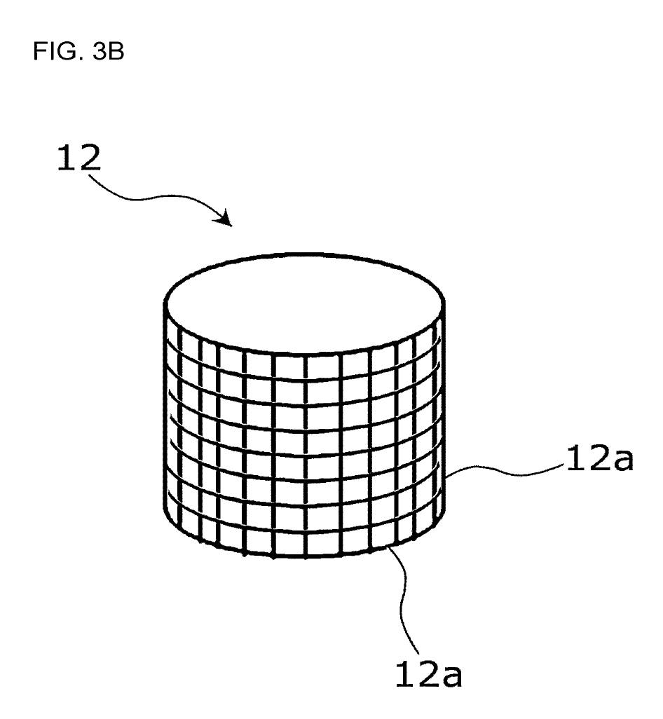

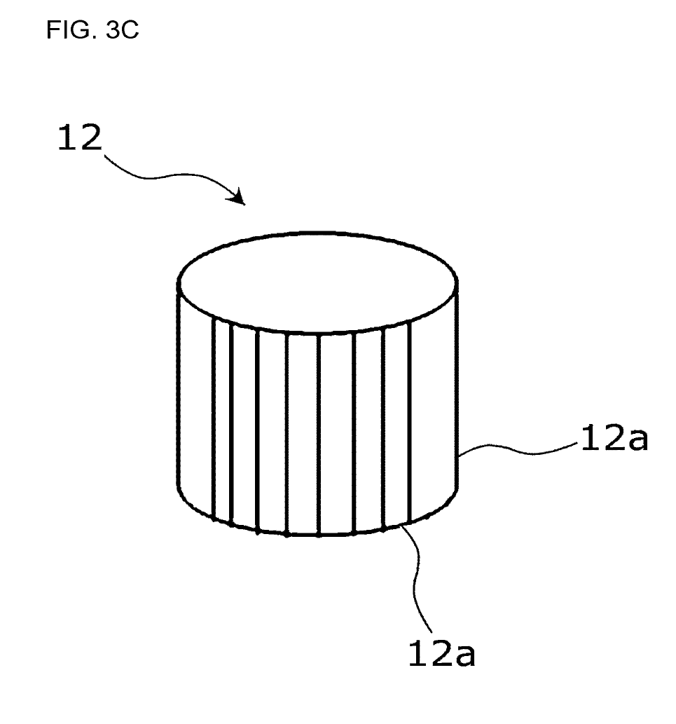

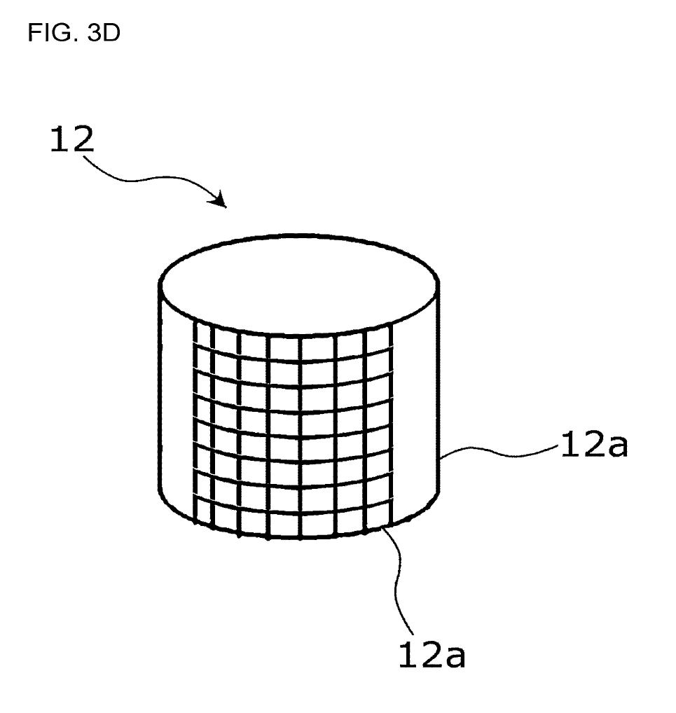

In the following, the structure of the end part 10 is described with reference to FIGS. 2A, 2B, and 3A. FIG. 2A is a schematic side view of the end part 10. FIG. 2B provides schematic cross sections taken along lines A-A′ and B-B′ in FIG. 2A, and illustrates the positional relationship of parts therein. In FIG. 2B, the cable 11, a direction controller 16, and a drive unit 18 are not illustrated. FIG. 3A is a schematic perspective view of an ultrasound transducer 12, which is a one-dimensional (1D) transducer array where ultrasound oscillators 12a are provided all over the outer peripheral surface of a support.

### (Overview of End Part)

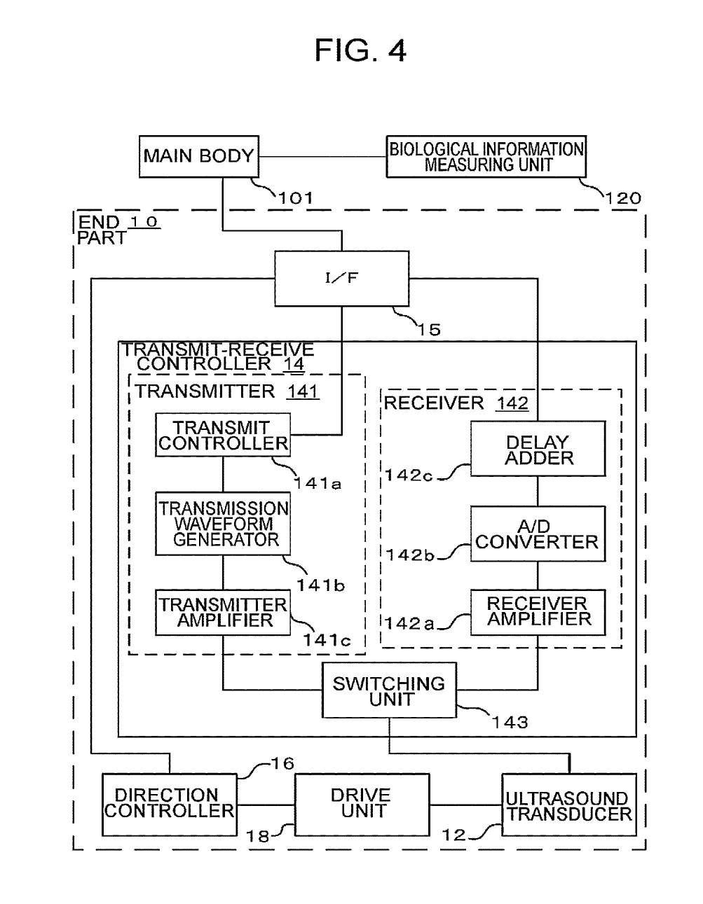

In the examples of FIGS. 1 and 2A, the end part 10 in a capsule form is used as a device for transmitting/receiving ultrasound waves. As illustrated in FIG. 2B, the end part 10 includes, in a container 10a that is formed in an ellipsoid, the ultrasound transducer 12, a transmit-receive controller 14, an interface (I/F) 15, and the like (see FIG. 4). Although not illustrated in FIG. 2B, the direction controller 16 and the drive unit 18 may be provided inside the container 10a.

As illustrated in FIG. 2B, in the ellipsoidally formed end part 10, for example, the cable 11 is connected to one longitudinal end of the container 10a. A power supply line for supplying power to the end part 10 and a signal line in the cable 11 run through the inside of the container 10a. These lines are connected to the transmit-receive controller 14, the direction controller 16, and the drive unit 18. As described below, when the container 10a is configured to be placed on tissue in the subject's body, the cable 11 can be configured to prevent the end part 10 from moving in the subject's body. For example, a part of the cable 11 may be fixed to a fixing part (not illustrated) that is fixed to a part of the tissue in the subject's body. Examples of the fixing part include a mouthpiece worn by the subject. By providing a mouthpiece with the fixing part, the extent to which the cable 11 is inserted into the subject's body can be kept within a predetermined range. Thus, the end part 10 can be fixed in the subject's body.

For another example, the container 10a of the end part 10 may be configured to expand so that it is appressed to the body tissue of the subject such as the esophagus. By appressing the container 10a to the body tissue, the end part 10 can be stayed inside the body. Although not illustrated, in such a configuration, the container 10a is formed to have a double-bag structure. The ultrasound transducer 12 is placed in the inner bag of the container 10a. The outer bag of the container 10a is connected to the cable 11. The cable 11 is communicated with the outer bag, so that fluid, i.e., liquid such as sterile water and the like, can be injected from a pipe 11c (see FIG. 2B) in the cable 11. The container 10a expands with the injection of fluid, and contracts when the fluid is discharged. While the ultrasound transducer 12 is provided in the container 10a of the end part 10, whether other elements, such as the transmit-receive controller 14, the direction controller 16, and the drive unit 18, are provided to the end part 10 is changed as appropriate depending on the structure of the ultrasound transducer 12 (element array, etc.).

### (Structure of Entire Ultrasound Transducer and Each Component)

The end part 10 illustrated in the example of FIG. 2B uses the ultrasound transducer 12 in which the rectangular ultrasound oscillators 12a are arranged in a circular array, i.e., 1D array (see FIG. 3A). In the ultrasound transducer 12, the ultrasound oscillators 12a are arranged all over the outer peripheral surface of the support (not illustrated). Hereinafter, the structure, where a backing layer, a piezoelectric element, a front electrode, a back electrode, and an acoustic matching layer on the support are arranged in layers, is referred to as the “ultrasound oscillators” 12a. In addition, a group of the support, the ultrasound oscillators 12a, and an acoustic lens 12c is referred to as the “ultrasound transducer” 12. The support (not illustrated) that supports the ultrasound oscillators 12a is, for example, formed in a cylinder, the inside of which is hollow along the central axis. The support may have a columnar form. If all the ultrasound oscillators 12a are required to be tilted to change the transmission direction of ultrasound waves (ultrasound beam angle, etc.), the support is connected to the drive unit 18. The ultrasound oscillators 12a are configured with the backing layer, the piezoelectric element, the front electrode, the back electrode, and the acoustic matching layer arranged radially in layers from the outer peripheral surface of the support toward the outside.

The piezoelectric element (not illustrated) is provided with the back electrode on a surface on the side of the backing layer (on the side of the support), and the front electrode on a surface on the opposite side (the side of the acoustic lens). The piezoelectric element converts a voltage applied to the front electrode and the back electrode into ultrasound waves. The ultrasound waves are transmitted to the subject. Having received reflected waves from the subject, the piezoelectric element converts the waves into voltage (echo signal). The piezoelectric element is generally made of such material as PZT (piezoelectric zirconate titanate/lead zirconate titanate/Pb(Zr, Ti)O₃). PVDF (polyvinylidene difluoride/polyvinylidene fluoride/(CH₂CF₂)ₙ) may also be used. The use of a PVDF film as a piezoelectric element facilitates making the end part 10 because of its flexibility. Further, the ultrasound oscillators 12a can be thinner in the layer direction, and thus the end part 10 can be downsized. Moreover, PVDF films possess good resistance to shock. As for other examples of the piezoelectric element, barium titanate (BaTiO₃), PZNT (Pb(Zn_{1/3}Nb_{2/3}) O₃—PbTiO₃) single crystal, PMNT (Pb(Mg_{1/3}Nb_{2/3}) O₃—PbTiO₃) single crystal, and the like may be used. The piezoelectric element may be of single layer, or it may be multilayered.

Part of all the piezoelectric elements may be used as pyroelectric elements and connected to a temperature sensing circuit (not illustrated). The temperature sensing circuit detects the temperature around the ultrasound oscillators 12a based on a pyroelectric voltage value or a pyroelectric current value received from the pyroelectric elements. The temperature sensing circuit may be located in the end part 10, or in the main body 101. Since the end part 10 is placed in the subject's body, it is effective in monitoring an observation site to enable the operator to know the temperature.

The acoustic matching layer is arranged adjacent to the acoustic lens 12c side in the front electrode of the piezoelectric element. That is, the acoustic matching layer is located between the piezoelectric element and the acoustic lens 12c. The acoustic matching layer matches acoustic impedance between the piezoelectric element and the subject. There may be two or more acoustic matching layers arranged in the layer direction. In this case, materials that vary in acoustic impedance in stages are used for the acoustic matching layers. This structure achieves acoustic matching by changing acoustic impedance in stages between the piezoelectric element and the acoustic lens 12c.

The backing layer is arranged adjacent to the support side in the back electrode of the piezoelectric element. The backing layer absorbs ultrasound waves emitted to the opposite direction to their irradiation direction (backward) during ultrasound beam transmission, thereby suppressing the excessive oscillation of the piezoelectric element. The backing layer suppresses the reflection from the back surface of the piezoelectric element that is oscillating. Therefore, with the backing layer, it is possible to avoid adverse effect on transmitting/receiving of ultrasound beam pulses. As the backing layer, in view of acoustic attenuation, acoustic impedance, and the like, any materials such as an epoxy resin containing PZT powder, tungsten powder, etc., rubber filled with polyvinyl chloride and/or ferrite powder, or porous ceramic impregnated with resin such as epoxy, and the like may be used.

### <Acoustic Lens>

The acoustic lens 12c (see FIG. 2B) converges transmitted/received ultrasound waves and forms them into a beam shape. The acoustic lens 12c is made of such material as silicone having an acoustic impedance similar to the living body. If the ultrasound oscillators 12a are in a 2D array, and capable of converging ultrasound waves into a beam by electronic scanning, the acoustic lens 12c may not be included.

When the end part 10 is inserted from the esophagus of the subject and the transmission direction of ultrasound waves is pointed to the heart, a wedge-shaped offset 12f may be added between the acoustic lens 12c and the ultrasound oscillators 12a as illustrated in FIG. 2C. This makes the acoustic lens 12c tilted to the support of the ultrasound oscillators 12a. With this structure, directions of ultrasound waves from the piezoelectric elements are converged into a different direction. Depending on the tilt angle of the offset 12f, it becomes unnecessary to perform drive control for transmitting ultrasound waves from the ultrasound oscillators 12a of the end part 10 placed in the esophagus to the heart, or the drive control can be simplified.

In the structure illustrated in FIG. 3A, in response to an instruction signal on the transmission direction of ultrasound waves from the main body 101, the direction controller 16 and the drive unit 18 (described later) tilt the ultrasound transducer 12 to adjust the transmission direction. If the offset 12f is provided, the tilting operation may not be necessary.

### (Other Examples of Ultrasound Transducer)

Referring to FIGS. 3B to 3D, other examples of the structure of the ultrasound transducer 12 are described. FIGS. 3B to 3D each illustrate a schematic perspective view of the ultrasound transducer 12. FIG. 3C illustrates the 1D array ultrasound transducer 12, while FIGS. 3B and 3D illustrate the 2D array ultrasound transducer 12. Besides, FIG. 3B illustrates the ultrasound transducer 12, in which the ultrasound oscillators 12a are provided all over the outer peripheral surface of the support. FIGS. 3C and 3D illustrate the ultrasound transducer 12, in which the ultrasound oscillators 12a are provided to a part of the outer peripheral surface of the support.

In the example of FIG. 3B, the ultrasound oscillators 12a are arranged in a 2D array all over the outer peripheral surface of the support. In this structure, it is possible to switch the elements to be driven by the transmit-receive controller 14 (described later), and deflect and converge ultrasound waves (ultrasound beams) by electronic scanning. In the ultrasound transducer 12 illustrated in FIG. 3B, it is possible to deflect and converge ultrasound waves, by electronic scanning, not only in a direction in which the elements are arrayed (azimuth direction), but also in the elevation direction substantially perpendicular to the direction. Accordingly, there may be no need to rotate and tilt the ultrasound transducer 12. In this case, the structure does not include the direction controller 16 and the drive unit 18. The acoustic lens 12c may also not be included.

In the example of FIG. 3C, the ultrasound oscillators 12a are arranged in a 1D array in a part in the circumferential direction of the outer peripheral surface of the support. That is, for example, when the support is of a cylindrical form, the ultrasound oscillators 12a are arrayed in an area within a predetermined angle range (e.g., 60°) from the central axis. In this structure, upon receipt of an instruction signal from the main body 101, the direction controller 16 and the drive unit (described later) perform rotating or tilting of the ultrasound transducer 12, or both.

In the example of FIG. 3D, the ultrasound oscillators 12a are arranged in a 2D array in a part in the circumferential direction of the outer peripheral surface of the support. In this structure, upon receipt of an instruction signal from the main body 101, the direction controller 16 and the drive unit 18 (described later) rotate the ultrasound transducer 12. The condition where the ultrasound oscillators 12a are arrayed in a part means that, for example, when the support is of a cylindrical form, the ultrasound oscillators 12a are arrayed in the azimuth and elevation directions in an area within a predetermined angle range (e.g., 60°) from the central axis.

### (Modification of End Part)

If the used as the piezoelectric element is among those having low acoustic impedance such as PVDF, the backing layer may be configured to reflect ultrasound waves radiated thereto instead of absorbing them. For example, a material that doubles as the backing layer and the support of the ultrasound oscillators 12a may be used. The use of a shape-memory alloy for the backing layer enables the use of the end part 10 having the following structure. The modification of the end part 10 is described below with reference to FIG. 2D.

The container 10a is configured such that the entire end part 10 is contracted when inserted into the subject's body. As illustrated in FIG. 2D, layers from the acoustic matching layer to the piezoelectric element are arranged on a flexible printed circuit (FPC) board 12d. On the FPC board 12d may be arranged an integrated circuit (IC) 12e having the function of the transmit-receive controller 14 and the like. The transmit-receive controller 14 is electrically connected to the electrode of the piezoelectric element via a pattern formed on the FPC board 12d and the like. The FPC board 12d is formed on the backing layer made of a shape-memory alloy.

The container 10a is configured such that, having been inserted in the subject's body, for example, when placed in the esophagus, the entire end part 10 is expanded by the injection of liquid such as water and the like through the cable 11 (see FIG. 2B). When the container 10a is expanded, a predetermined space is formed therein. The shape-memory alloy as the backing layer is configured to recover, for example, cylindrical or columnar form as illustrated in FIG. 3A, when being expanded. By the discharge (suction, etc.) of liquid injected in the container 10a, the entire end part 10 is contracted.

The ultrasound transducer 12 is supported by the FPC board 12d and the backing layer made of a shape-memory alloy. Accordingly, in response to the contraction of the container 10a, the entire ultrasound transducer 12 is also contracted. With this structure, the end part 10 becomes smaller when being contracted. Thus, the operator can arbitrarily expand/contract the end part 10, and thereby can easily insert and remove the end part 10 into/from the subject's body.

### (Transmit-Receive Controller)

Next, referring to FIG. 4, a description is given of the transmit-receive controller 14 of the end part 10. FIG. 4 is a schematic block diagram illustrating an example of the functional structure of the end part 10 of the ultrasound diagnosis apparatus 100 of the first embodiment. As illustrated in FIG. 4, the transmit-receive controller 14 includes a transmitter 141, a receiver 142, and a switching unit 143. They are each described below.

### (Transmitter)

The transmitter 141 of the end part 10 includes a transmit controller 141a, a transmission waveform generator 141b, and a transmitter amplifier 141c. The transmitter 141 receives an instruction signal on the transmission of ultrasound waves from the main body 101 (a transmitter/receiver unit 105 or the like, see FIG. 5) through the I/F 15. The transmitter 141 further includes a clock generation circuit, a transmitter delay circuit (not illustrated), and the like controlled by the transmit controller 141a. The clock generation circuit generates clock signals for determining the transmission frequency and the transmission timing of ultrasound waves. For example, the clock generation circuit feeds the transmitter delay circuit with a reference clock signal. The transmitter delay circuit sends the transmission waveform generator 141b a drive signal having a predetermined delay time. The predetermined delay time is determined based on the transmission focal point of ultrasound waves.

The transmission waveform generator 141b includes, for example, a pulser circuit (not illustrated). The pulser circuit includes therein as many pulsers as individual channels corresponding to the ultrasound oscillators 12a, and generates transmission drive pulses. The pulser circuit repeatedly generates a rate pulse at a predetermined pulse repetition frequency (PRF). The rate pulses are distributed into the number of the channels, and sent to the transmitter delay circuit.

The transmitter delay circuit of the transmit controller 141a provides the rate pulse with a transmission delay time related to the transmission direction and the transmission focus. Transmission drive pulses are generated at timing based on the rate pulses each being delayed. The transmission drive pulses are amplified by the transmitter amplifier 141c, and sent to the switching unit 143. As described above, the transmitter delay circuit provides the pulser circuit with a delay time to focus ultrasound waves for transmission, thereby converging the ultrasound waves into a beam. With this, the transmission directivity of the ultrasound waves is determined. In addition, the transmitter delay circuit changes the transmission delay time to be given to each rate pulse, thereby controlling the transmission direction of ultrasound waves from the ultrasound wavefronts of the ultrasound oscillators 12a.

### (Switching Unit)

The switching unit 143 has a switch relating to transmitting/receiving of ultrasound waves, and controls switching between the transmitter 141 and the receiver 142. If scan mode on the main body 101 side is set to continuous wave Doppler (CWD) mode, as described below, the switching unit 143 connects some elements of the ultrasound oscillators 12a to the transmitter 141 for transmission, and connects some others to the receiver 142 for reception.

If the scan mode on the main body 101 side is set to perform B (brightness) mode and pulsed wave Doppler (PWD) mode in parallel, the switching unit 143 alternately repeats control to sequentially switch elements to be driven according to the B mode and control to switch to elements that transmit ultrasound waves toward a set sample volume (sampling gate). In the B mode, a group of elements to be driven are shifted in the element array direction to control the transmission direction of ultrasound waves or the like.

Besides, the switching unit 143 switches sub-arrays each including a group of elements in m rows×n columns (a group of oscillators) in the 2D array ultrasound transducer 12. A transmission drive pulse from the transmitter amplifier 141c is applied to each element of the sub-array connected to the switch of the switching unit 143, and the piezoelectric element is driven.

### (Receiver)

The receiver 142 of the end part 10 receives echo signals corresponding to ultrasound waves reflected from the subject. The receiver 142 amplifies the echo signals received by the ultrasound transducer 12, and also adds delay thereto. By the delay addition of the receiver 142, the analog echo signals are converted to digital data having been subjected to phasing (i.e., subjected to beam forming). Specific examples are as follows.

The receiver 142 includes a receiver amplifier 142a, an A/D converter 142b, and a delay adder 142c. The receiver 142 may further include a sub-array delay adder (not illustrated). The receiver amplifier 142a amplifies echo signals received from the ultrasound transducer 12 with respect to each receiver channel. The A/D converter 142b converts the amplified echo signals to digital signals. Having been converted into digital signals, the echo signals are each stored in a digital memory (not illustrated). The digital memory is provided for each channel (or each element). Each echo signal is stored in the corresponding memory. The echo signal is also stored in an address corresponding to the time it is received. The A/D converter 142b is capable of thinning out data that has been filtered according to the bandwidth of the echo signal. If the receiver 142 has the sub-array delay adder (not illustrated), the sub-array delay adder can add echo signals from adjacent elements in the ultrasound oscillators 12a.

The delay adder 142c provides the echo signals each converted into a digital signal with a delay time required to determine the reception directivity. The reception delay time is calculated for each element. The delay adder 142c adds up the echo signals having the delay time. The delay adder 142c reads each of the echo signals from the digital memory as appropriate based on the required delay time calculated, and adds up them. The delay adder 142c repeats this addition while changing a reception focus position along the transmission beam. The addition emphasizes a reflection component from a direction corresponding to the reception directivity. The received beam signal processed by the receiver 142 is sent to a signal processor (a B-mode signal processing unit 107, a Doppler signal processing unit 108) via the I/F 15, the transmitter/receiver unit 105, or the like.

### (Direction Controller, Drive Unit)

In response to an instruction signal on the transmission direction of ultrasound waves from the main body 101, the direction controller 16 controls the drive unit 18. For example, the direction controller 16 drives the drive unit 18 to change the angle or orientation of the wavefront of the ultrasound waves according to ROI (Region of Interest) set on the main body 101 side. The drive unit 18 is comprised of, for example, a micro-actuator such as an ultrasound beam motor. The drive unit 18 is driven under the control of the direction controller 16. The drive unit 18 is connected to the ultrasound transducer 12. With this structure, when the drive unit 18 is driven, the ultrasound transducer 12 is rotated or tilted. Thus, by driving the drive unit 18, the transmission direction of ultrasound waves can be changed in the ultrasound transducer 12.

### <Structure of Biological Information Measuring Unit>

In FIG. 5, the biological information measuring unit 120 is connected to the main body 101. The biological information measuring unit 120 generates information indicating the conditions of the subject such as a biological signal, and sends the generated information to the main body 101. Examples of the biological information measuring unit 120 include bioelectric equipment (electrocardiograph, electroencephalograph, electromyography, etc.), respiratory equipment (respiratory flow meters, electronic spirometers, respiratory resistance meters, etc.), and medical monitoring equipment (singular monitor (bedside monitor), multiple monitors (central monitor), etc.), and the like. The medical monitoring equipment is configured to monitor vital signs such as ECG, blood pressure, respiratory rate, body temperature, pulse rate, blood oxygen saturation, exhaled gas partial pressure, and the like. Although FIG. 5 illustrates the biological information measuring unit 120 that is located outside the main body 101, some part thereof may be arranged in the main body 101 so that the measurement is performed in the main body 101.

In the first embodiment, the main body 101 is configured to receive the analysis result of biological information (ECG waveform, etc.) from the biological information measuring unit 120. That is, the biological information measuring unit 120 is configured to analyze biological information in real time according to the settings, and sends the analysis result to the main body 101. Described below is an example in which biological information is ECG waveform, and the biological information measuring unit 120 analyzes the ECG waveform in real time. The biological information measuring unit 120 includes a member that, in contact with the subject, directly obtains ECG waveforms from the subject such as, for example, electrodes. For another example, the biological information measuring unit 120 may obtain ECG waveforms from an outside ECG and concentrate solely on the analysis.

The biological information measuring unit 120 performs automatic classification according to intervals between adjacent heartbeats, the features of each heartbeat waveform, or the like by an analyzer. In addition, the biological information measuring unit 120 stores each heartbeat waveform with such information as a serial number, classification, R-R interval to the previous heartbeat (time interval between R waves). As a specific example, having received an ECG signal from the subject, the biological information measuring unit 120 performs filtering. The filtering includes waveform shaping such as the removal of baseline drift and the removal of noise from the ECG signal. All ECG waveforms subjected to the filtering are stored in the storage unit or the like.

The biological information measuring unit 120 detects QRS complex by a known method from ECG waveforms corresponding to all heartbeats stored, calculates R-R interval to the previous heartbeat, or the like. The biological information measuring unit 120 stores in advance reference waveform data used for the classification of waveforms. A plurality of sets of reference waveform data is set for each category, and when a desired category (normal heartbeat, premature ventricular contraction, etc.) is selected, the biological information measuring unit 120 retrieves reference waveform data corresponding to the selection. The biological information measuring unit 120 obtains the similarity of all the stored heartbeats to the reference waveform data. If the similarity is equal to or higher than a threshold, a waveform having the high similarity is extracted as one corresponding to the selected category.

One example of the reference waveform data is reference waveform data which is a model of typical abnormal waveform. In comparison with the waveform data of the abnormal waveform, if a waveform is determined as having similarity higher than the threshold, it is extracted as a selected typical abnormal waveform. Besides, when the variation of R-R interval is equal to or more than a predetermined rate (e.g., 10%) and P wave cannot be identified, this may be analyzed as abnormal (atrial fibrillation). When having extracted the abnormal waveform by analysis, the biological information measuring unit 120 may send the main control unit 104 a trigger signal indicating the detection of abnormalities instead of ECG waveforms. Hereinafter, the trigger signal indicating the detection of abnormalities may be referred to as “abnormality detection trigger”.

As described above, the biological information measuring unit 120 of the first embodiment receives ECG waveforms of the subject in real time from the ECG by any method, and extracts a specific ECG waveform to send it to the main body 101. The main control unit 104 receives the specific ECG waveform extracted by the biological information measuring unit 120. The specific ECG waveform is not necessarily of one type. In the following, an example is mainly described in which the specific ECG waveform indicates abnormal tissue in the subject's body. The specific ECG waveform of this case may be referred to as “abnormality indication ECG waveform”. The “abnormality indication ECG waveform” is an example of a waveform based on non-periodic motion in a predetermined site that moves periodically (non-periodic ECG waveform). The main body 101 may have the function of performing the analysis of the biological information measuring unit 120. In this case, the biological information measuring unit 120 may simply send ECG waveforms to the main body 101 without having the function of the analysis.

### <Structure of Main Body>

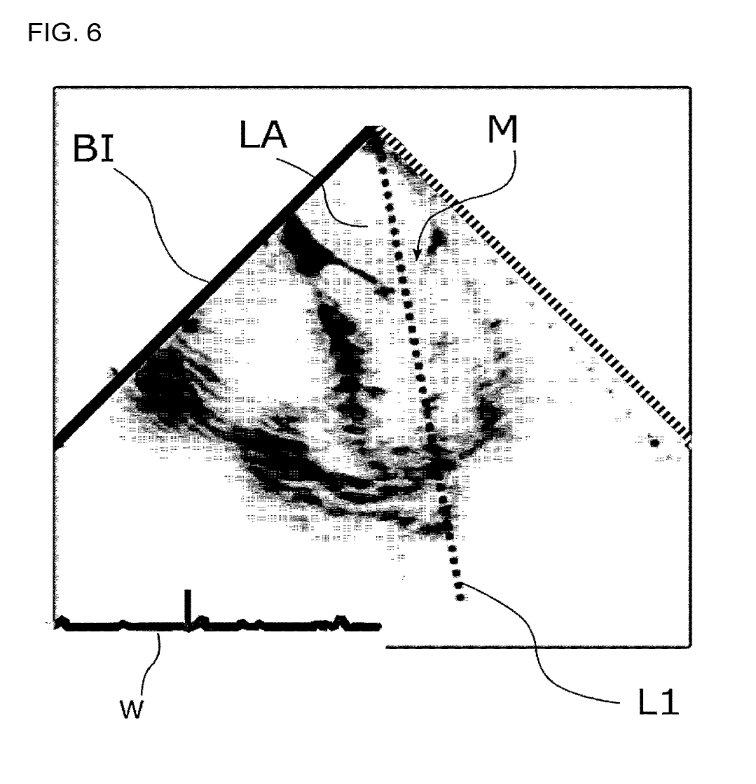

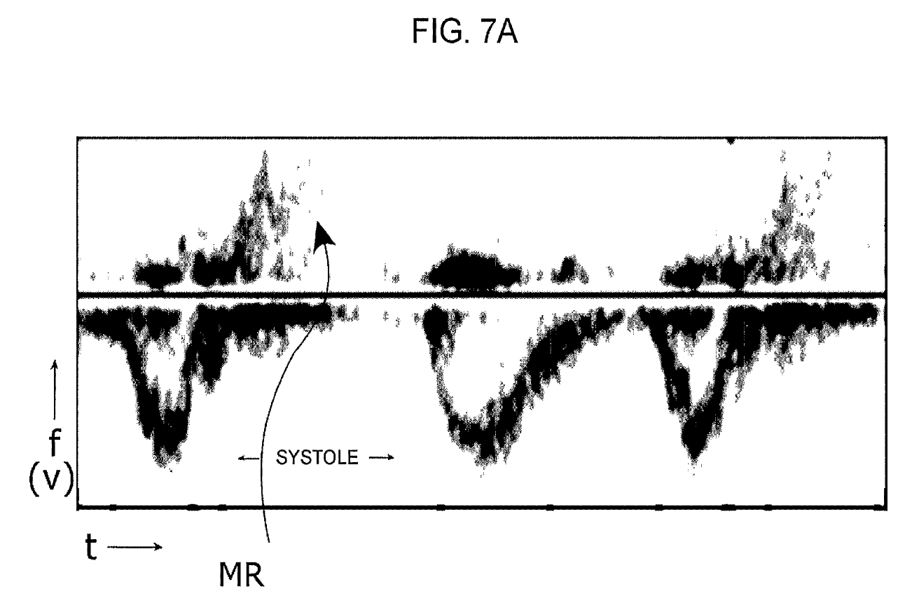

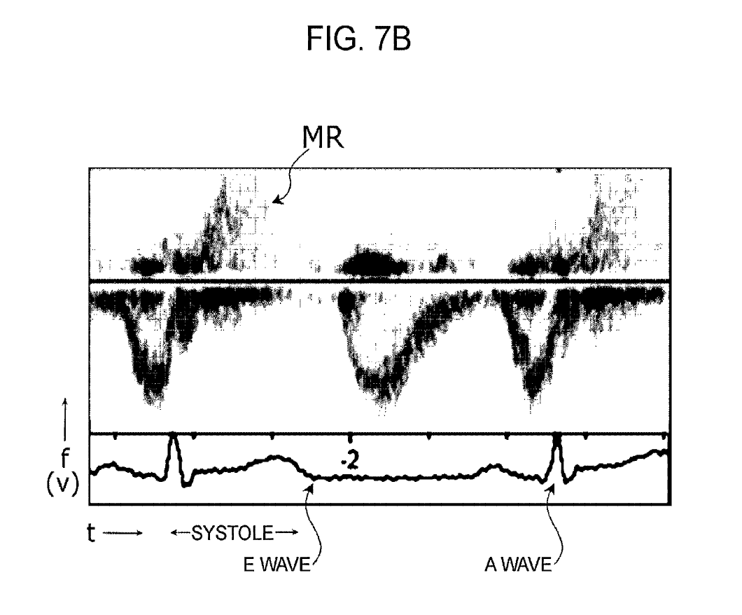

Next, the control and the operation of each part of the main body 101 are described with reference to FIG. 5. The ultrasound diagnosis apparatus 100 illustrated in FIG. 5 is, for example, used to obtain such images as those indicating the form of biological tissues such as the heart (see FIG. 6) and those indicating the state of blood flow (see FIG. 7A). As illustrated in FIG. 5, in the ultrasound diagnosis apparatus 100, the main body 101 is connected to the end part 10 and the biological information measuring unit 120. The end part 10 corresponds to an example of “ultrasound beam transmitter/receiver”. FIG. 5 is a schematic block diagram illustrating an example of the functional structure of the main body 101 of the ultrasound diagnosis apparatus 100 of the first embodiment.

The main body 101 includes therein units for performing input/output operations, calculations, controls, and the like of the ultrasound diagnosis apparatus 100 (see FIG. 5). In FIG. 5, the main body 101 includes, as the functional units, the operation unit 102, the display unit 103, the main control unit 104, the transmitter/receiver unit 105, the B-mode signal processing unit 107, the Doppler signal processing unit 108, a generating unit 109, and a direction setting unit 110. Incidentally, the biological information measuring unit 120 may be included in the configuration of the ultrasound diagnosis apparatus 100. The main body 101 may include a power supply connected to the end part 10 via the cable 11.

### (Operation Unit)

In response to operation by the operator, the operation unit 102 feeds signals and information corresponding to the operation to each unit. Examples of the operation unit 102 are not limited to a keyboard and a pointing device such as a mouse, but include any other user interfaces. For example, the input function of the operation unit 102 may be implemented as a software keyboard (softkey) on the touch panel integrated with the display unit 103. The operation unit 102 may have a function of receiving input of signals and information via media and networks. Note that, in the following, the ultrasound image includes not only anatomical images such as B-mode images but also waveform images based on the motion information of tissues and blood flow and color display images of brightness and color based on the motion information of tissues and blood flow.

If, for example, the operator operates a FREEZE button or an end button on the operation unit 102, transmitting/receiving of ultrasound waves is terminated or paused. Through the operation unit 102, the operator can set the number of heartbeats to capture images in the control of intermittent imaging (described later). The setting information is stored in a storage unit (not illustrated) in the transmitter/receiver unit 105. Using the operation unit 102, the operator can determine the initial settings such as scan mode for transmitting/receiving ultrasound waves. The operator can also specify sample volume (sampling gate) in Doppler mode through the operation unit 102. Further, the operator can determine the settings for monitoring biological information such as cardiac ejection fraction through the operation unit 102.

### (Display Unit)

The display unit 103 displays ultrasound images as well as operation screens, setting screens, and the like. Examples of the display unit 103 include any display devices such as a cathode ray tube (CRT), a liquid crystal display (LCD), a plasma display panel, an organic electroluminescent display (OELD), a field emission display (FED), and the like.

### (Main Control Unit)

The main control unit 104 includes a central processing unit (CPU), a read only memory (ROM), a random access memory (RAM), and the like. The CPU loads a control program into the RAM as appropriate, thereby implementing the functions of the main control unit 104. The main control unit 104 controls each part in the main body 101 as follows.

### (Transmitter/Receiver Unit; Transmitter Unit)

The transmitter/receiver unit 105 of the main body 101 transmits a signal (drive signal) related to the driving of the ultrasound transducer 12 to the transmit-receive controller 14 of the end part 10 according to selected scan mode. However, the drive signal is transmitted in response to an abnormality detection trigger or a specific ECG waveform extracted by the biological information measuring unit 120. For example, the main control unit 104 receives a specific ECG waveform from the biological information measuring unit 120. In response to the specific ECG waveform or an abnormality detection trigger, the main control unit 104 sends a trigger signal to the transmitter/receiver unit 105. Note that, in the main body 101, as well as the specific ECG waveform, ECG waveform obtained in real time may be displayed on the display unit 103. Therefore, upon receipt of the specific ECG waveform, the main control unit 104 sends a trigger signal to the transmitter/receiver unit 105.

Having received the trigger signal from the main control unit 104, the transmitter unit of the transmitter/receiver unit 105 sends the end part 10 a signal related to the driving of the ultrasound transducer to obtain ultrasound images for the predetermined number of heartbeats. The predetermined number of heartbeats is set in advance by the operator or the like when the ultrasound diagnosis apparatus 100 starts monitoring the subject, or before or after it. To capture images for the number of heartbeats set as above, the transmitter/receiver unit 105 obtains ECG waveforms in real time from the biological information measuring unit 120.

When capturing images in predetermined cardiac time phase, the main control unit 104 receives ECG waveform in the predetermined cardiac time phase extracted by the biological information measuring unit 120. Based on the ECG waveform in the predetermined cardiac time phase, the main control unit 104 sends a trigger signal to the transmitter/receiver unit 105. At the start of monitoring, regardless of whether a trigger signal has been received from the main control unit 104, ultrasound waves may be transmitted for the predetermined number of heartbeats.

For example, the main control unit 104 receives a selection operation of scan mode (scan sequence) through the operation unit 102. In response to this operation, the main control unit 104 controls the transmitter/receiver unit 105 depending on the selected scan mode. According to the selected scan mode, transmission frequency, transmission driving voltage, and the like are changed. Examples of the scan mode include B-mode, power Doppler mode (PDI; Power Doppler Imaging), pulsed Doppler mode, continuous wave Doppler mode, color Doppler mode (CDI; Color Doppler Imaging/CFM; Color Flow Mapping), tissue Doppler mode (TDI; Tissue Doppler Imaging), M (motion) mode, and the like. In addition, any combination of them is also selectable for the scan mode.

### (Transmitter/Receiver Unit; Receiver Unit)

From the end part 10, the transmitter/receiver unit 105 of the main body 101 receives a digital echo signal subjected to predetermined processing by the transmitter 141. The echo signal is sent to the signal processor (the B-mode signal processing unit 107, the Doppler signal processing unit 108).

### (Signal Processor; B-Mode Signal Processing Unit)

The signal processor includes the B-mode signal processing unit 107 and the Doppler signal processing unit 108. Having received the signal from the receiver unit of the transmitter/receiver unit 105, the B-mode signal processing unit 107 creates a visual image of amplitude information of the signal. Specifically, the B-mode signal processing unit 107 performs band-pass filtering on the received beam signal, then detects the envelope of the received beam signal, and compresses detected data by logarithmic transformation. Thus, the B-mode signal processing unit 107 generates RAW data of a B-mode image.

### (Signal Processor; Doppler Signal Processing Unit)

As Doppler processing, the Doppler signal processing unit 108 removes Doppler shift frequency component by phase detection of the received beam signals, and performs fast Fourier transform (FFT). The Doppler signal processing unit 108 extracts a Doppler shift by the frequency analysis of the received beam signal (Doppler signal). The Doppler signal processing unit 108 extracts, based on the Doppler shift, contrast medium echo component as well as blood flow and tissues caused by Doppler effect, and generates RAW data of a Doppler image extracting moving object information such as average velocity, variance, and power with respect to a plurality of points.

The Doppler signal processing unit 108 may be configured to perform color Doppler processing. The blood flow and tissue motion information is visualized by the color Doppler processing. The blood flow and tissue motion information includes velocity, distribution, and power. For example, the Doppler signal processing unit 108 processes the received beam signal, thereby generating RAW data of a color flow mapping (CFM) image in the region of interest. In particular, the Doppler signal processing unit 108 performs quadrature detection of the received beam signal from the receiver unit of the transmitter/receiver unit 105. The Doppler signal processing unit 108 then performs frequency analysis on the echo signal after the quadrature detection by autocorrelation method. By the frequency analysis, the Doppler signal processing unit 108 calculates the variance and the average velocity of blood flow at each point of the sample. The Doppler signal processing unit 108 generates the RAW data of the color flow mapping image representing the calculated variance and the average flow velocity by color. The Doppler signal processing unit 108 also calculates the power of blood flow based on the received beam signal subjected to the quadrature detection. The Doppler signal processing unit 108 generates the RAW data of the color flow mapping image representing the calculated power by color.

The signal processing units send the RAW data (ultrasound beam raster data) subjected to the signal processing to the generating unit 109. Incidentally, the B-mode signal processing unit 107 and the Doppler signal processing unit 108 of the embodiment can process both two-dimensional echo data and three-dimensional echo data.

### (Generating Unit)

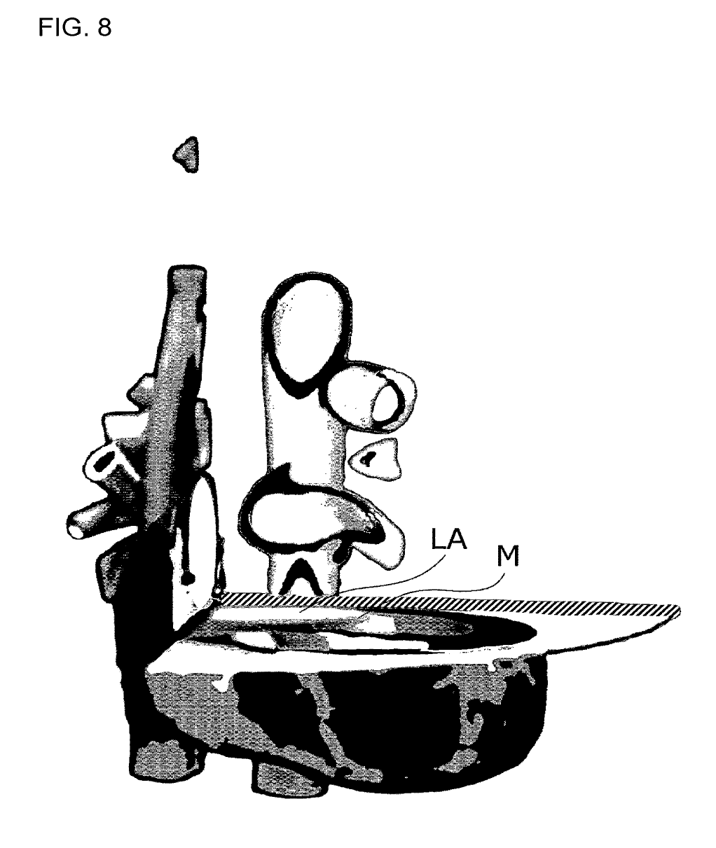

With reference to FIGS. 6, 7A, 7B, and 8, the operation of the generating unit 109 is described. FIG. 6 is a schematic diagram of an example of a B-mode image BI generated by the generating unit 109 of the first embodiment. FIG. 7A is a schematic diagram of an example of a Doppler spectrum image generated by the generating unit 109. FIG. 7B is a schematic diagram of an example of the Doppler spectrum image illustrated in FIG. 7A displayed in parallel with ECG waveform fed by the biological information measuring unit 120. FIG. 8 is a schematic diagram of screen data illustrating a positional relationship for obtaining the cross section of the B-mode image BI illustrated in FIG. 6. Having received RAW data based on echo signals for the predetermined number of heartbeats, the generating unit 109 generates ultrasound image data corresponding to the number of heartbeats.

The generating unit 109 generates ultrasound image data based on the RAW data after the signal processing output from the signal processor (the B-mode signal processing unit 107, the Doppler signal processing unit 108). The generating unit 109 includes, for example, DSC (Digital Scan Converter). The generating unit 109 converts the RAW data subjected to the signal processing represented by a signal sequence of a scan line into image data represented by a Cartesian coordinate system (scan conversion). For example, by applying the scan conversion to the RAW data subjected to the signal processing by the B-mode signal processing unit 107, the generating unit 109 generates B-mode image data representing signal strength by brightness for each form of the tissues of the subject (see FIG. 6). As illustrated in FIG. 8, FIG. 6 is a four-chamber cross-sectional view, approached from the esophagus. FIG. 6 illustrates left atrium LA, mitral valve M, and a broken line L1 indicating the transmission direction of ultrasound waves. FIG. 6 also illustrates ECG waveform W.

Besides, the generating unit 109 performs coordinate transformation on the RAW data having undergone the color Doppler processing or the Doppler processing, and generates data of the Doppler image and data of the color flow mapping image that can be displayed on the display unit 103. For example, based on the result of the frequency analysis of the Doppler signal (echo signal) using FFT (Fast Fourier Transform) by the Doppler signal processing unit 108, the generating unit 109 generates a Doppler spectrum image where the velocity information of the moving object (velocity information of blood flow, tissues, etc.) is drawn along the time series (see FIG. 7A).

In FIG. 7A, the vertical axis indicates frequency f (velocity v), while the horizontal axis indicates time t, and thus the spectrum is represented (FFT display). Additionally, in the waveform display, the crest value represents the magnitude of the velocity, and the brightness represents the strength of the Doppler spectrum (corresponding to the power of the Doppler signal). In FIG. 7A, the tone is displayed in reverse to enhance the viewability of the image (the same is applied to FIG. 7B).

After ultrasound waves are transmitted/received with time through the end part 10, Doppler spectrum images are sequentially generated by the generating unit 109 through the above processing. The display unit 103 sequentially displays the generated images, and thus the state that the frequency f (the velocity v of the object) changes from moment to moment is displayed as a pattern.

The generating unit 109 can obtain ECG waveform from the biological information measuring unit 120 connected to the main body 101 via the main control unit 104. As illustrated in FIG. 7B, based on the acquired ECG waveform, the generating unit 109 generates an image capable of representing a Doppler spectrum image and the ECG waveform synchronously in parallel.

Also, for example, from the RAW data of a color flow mapping image, the generating unit 109 generates color flow mapping images as an average velocity image indicating moving object information (blood flow information, tissue motion information, etc.) a variance image, a power image, and a combination of these. The generating unit 109 may generate a composite image by combining any images from the B-mode image BI (see FIG. 6), the color flow mapping image, and the Doppler image. For example, the generating unit 109 generates a color flow mapping image by overlaying a color display based on the motion information of tissues and blood flow on the B-mode image BI (or MPR (Multi-Planar Reconstruction) image) as well as generating a Doppler spectrum image by pulsed Doppler mode. Further, based on ECG waveform obtained from the biological information measuring unit 120, the generating unit 109 can generate an image capable of representing the color flow mapping image and the Doppler spectrum image in parallel with the ECG waveform.

When a volume data processing unit (not illustrated) is provided to the signal processor of the main body 101, the generating unit 109 may also display a volume rendering image and an MPR image. In this case, based on the echo signal received by the ultrasound transducer 12, the signal processor generates volume data representing the three-dimensional shape of tissues in the subject's body directly from the RAW data, or image data generated by a digital scan converter. Having obtained the volume data from the signal processor, the generating unit 109 generates a volume rendering image. The generating unit 109 can also generate MPR images from the volume data.

### (Direction Setting Unit)

The direction setting unit 110 sets the transmission direction of ultrasound waves by the ultrasound transducer 12 in the end part 10. The transmission direction is set based on operator's operation on the operation unit 102 or transmission direction data received from a search unit 111 (described later). The direction setting unit 110 sends the transmit-receive controller 14 or the direction controller 16 of the end part 10 determined transmission direction data. The direction setting unit 110 includes a storage unit (not illustrated) to store sample volume and the transmission direction data.

With respect to the setting of the transmission direction of ultrasound waves, the direction setting unit 110 receives such operations as selecting scan mode, setting sample volume, rotating/tilting the ultrasound transducer 12, and the like. The direction setting unit 110 also sets elements (or channels) to apply a drive signal in the ultrasound transducer 12 of the end part 10 depending on scan mode (continuous wave Doppler mode, etc.).

The information set for the transmission direction of ultrasound waves according to scan mode selection and sample volume setting (elements to be driven, angle/direction with respect to the ultrasound wavefront, etc.) is sent to the transmit-receive controller 14 of the end part 10 via the transmitter/receiver unit 105. The information set for the transmission direction of ultrasound waves according to the rotation/tilting of the ultrasound transducer 12 (the amount of rotation, the tilt angle of the ultrasound transducer 12, etc.) is sent to the direction controller 16 of the end part 10. Incidentally, the direction setting unit 110 corresponds to an example of a “changer”. Besides, in combination with the direction controller 16 and the drive unit 18 of the end part 10, the direction setting unit 110 corresponds to an example of a “changer”. In combination with the transmitter/receiver unit 105 and the transmit-receive controller 14 of the end part 10, the direction setting unit 110 corresponds to an example of “changer”.

### <Operation>

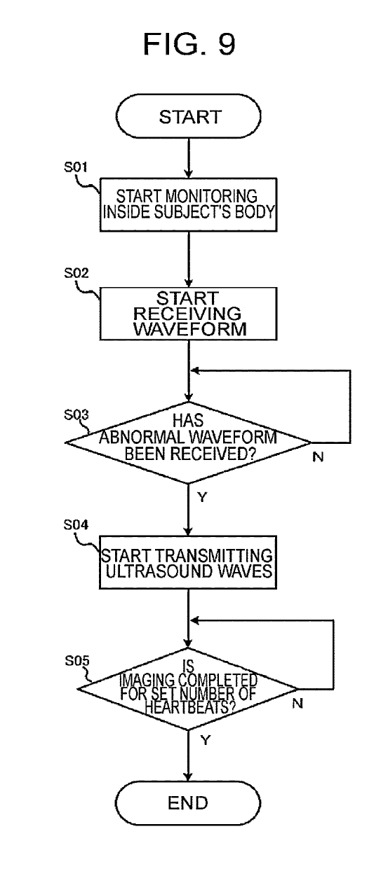

In the following, a description is given of a control flow to perform the intermittent imaging according to the embodiment with reference to FIG. 9. FIG. 9 is a flowchart schematically illustrating the operation of the ultrasound diagnosis apparatus 100 of the first embodiment.

### (Step S01)

When the operator has determined the initial setting, the monitoring of body tissue is started. The initial setting is made through the operation unit 102, and includes selection of scan mode, settings of transmission focal point, sample volume, and the like. The initial setting also includes the condition where the biological information measuring unit 120 can obtain the biological information of the subject. For example, setting of ECG, setting of the analyzer of the biological information measuring unit 120, and the like are required. The initial setting further includes the condition where the end part 10 is inserted in the subject's body, and positions of the end part 10 and the tissue to be observed are adjusted. Besides, the operator sets the number of heartbeats as a reference for intermittently capturing ultrasound images.

### (Step S02)

Having started measuring ECG waveform, the biological information measuring unit 120 sends the ECG waveform to the main control unit 104. Depending on the settings provided in advance in the biological information measuring unit 120, the biological information measuring unit 120 may send the main control unit 104 a specific ECG waveform (R wave, T wave, etc.) extracted by analyzing the obtained ECG waveform.

### (Step S03)

The main control unit 104 determines whether an ECG waveform indicating abnormality (abnormality detection trigger, abnormal waveform) has been received from the biological information measuring unit 120. If, in step S03, determining that an ECG waveform indicating abnormality has not been received (step S03; No), the main control unit 104 repeats this determination.

### (Step S04)

In step S03, having determined that an ECG waveform indicating abnormality has been received (step S03; Yes), the main control unit 104 sends a trigger signal to the transmitter/receiver unit 105. Upon receipt of the trigger signal, the transmitter/receiver unit 105 reads data of the number of heartbeats set in advance for the intermittent imaging from the storage unit (not illustrated). Having retrieved the data of the number of heartbeats, the transmitter/receiver unit 105 receives, from the main control unit 104, ECG waveform obtained in real time by the biological information measuring unit 120. Based on the real-time ECG waveform, the transmitter/receiver unit 105 makes the end part 10 start transmitting/receiving ultrasound waves according to the timing representing a specific waveform (R wave, etc.).

### (Step S05)

When the end part 10 starts transmitting/receiving ultrasound waves in step S04, the transmitter/receiver unit 105 receives an echo signal, and the generating unit 109 generates an ultrasound image through several types of signal processing. Having started receiving ECG waveforms as well as transmitting ultrasound waves, the transmitter/receiver unit 105 starts measuring the timing to terminate the intermittent imaging from these time points. That is, the transmitter/receiver unit 105 determines whether the imaging is completed for the number of heartbeats set in advance based on, for example, ECG waveform received in real time. When the imaging has not yet been completed (step S05; No), the transmitter/receiver unit 105 repeats this determination.

For another example, the main control unit 104 may be configured to obtain an elapsed time for one heartbeat from ECG waveforms being received in real time. That is, the main control unit 104 obtains the imaging time for a predetermined number of heartbeats based on the elapsed time for one heartbeat. Having determined that the imaging for the predetermined number of heartbeats is completed, the main control unit 104 sends a trigger indicating the end of imaging to the transmitter/receiver unit 105.

When determining that the intermittent imaging is completed (step S05; Yes), the transmitter/receiver unit 105 terminates the intermittent imaging without transmitting a signal related to the driving of the ultrasound transducer 12 to the end part 10.

In the ultrasound diagnosis apparatus 100 of this embodiment, when the initial setting is determined by the operator and the monitoring of body tissue is started, the main control unit 104 does not immediately issue a trigger signal (abnormality detection trigger), and also the transmitter/receiver unit 105 does not send the end part 10 a signal related to the driving of the ultrasound transducer 12. In this embodiment, the imaging of body tissue is initiated when the main control unit 104 receives a specific ECG waveform or an abnormality detection trigger from the biological information measuring unit 120. Further, after the start of the transmission of ultrasound waves, upon completion of the imaging for the number of heartbeats set in advance, the transmitter/receiver unit 105 of the main body 101 terminates the intermittent imaging. With this structure, ultrasound waves can be prevented from being transmitted continuously and all the time in the subject's body. Accordingly, it is possible to avoid heat generation due to the prolonged transmission of ultrasound waves.

Moreover, the ultrasound diagnosis apparatus 100 is configured to acquire ultrasound images while looking for the right time to capture images, such as when there is a change in the conditions of the subject informed from the biological information measuring unit 120. That is, the ultrasound diagnostic apparatus 100 does not continue to acquire ultrasound images if the conditions of the subject are stable for a long period of time. As a result, the viewer of ultrasound images in the monitoring is not forced to examine unnecessary images. Thus, it is possible to reduce the burden on the viewer. This results in the improved efficiency of the ultrasound examination.

The ultrasound diagnosis apparatus 100 of this embodiment includes the end part 10 having a structure in which the ultrasound transducer 12 is housed in the container 10a in a capsule form. In this respect, the main body 101 may be referred to as an external unit with respect to the container 10a. The end part 10 is inserted in the subject's body. On the other hand, if a transesophageal echocardiography (TEE) probe is inserted in the esophagus, the guide tube portion form the grip to the end part stays in the esophagus. For example, when ultrasound waves are transmitted and received between a predetermined position in the esophagus and the heart, the guide tube portion is placed in the esophagus while at least ultrasound waves are being transmitted and received. That is, while an observation site such as the heart or the like is being monitored, the guide tube portion to the end part stays in the esophagus of the subject all the time.

The guide tube portion and the end part of the TEE probe are provided therein with not only a signal line for exchanging signals with the ultrasound transducer and a power supply or the like, but also a wire for bending the end part. This means that the subject is obliged to bear with patience the guide tube portion or the like that includes therein a wire and the like being placed in the esophagus. If the monitoring continues for a long time, it may impose a burden on the subject depending on his/her condition. As a result, the TEE probe may not be used for the continuous monitoring of the observation site. If ultrasound waves are transmitted and received at the outside of the body to avoid this problem, it is required to consider the influence of tissues (bones, lungs, etc.) present in the transmission/reception directions of waveforms. As in the embodiment, if the end part 10 is in a capsule form, and only minimal lines such as a signal line and a power supply line are passed through the cable 11, it is possible to reduce the burden on the subject compared to the case of using the TEE probe.

### <Modification 1>

In the following, a modification 1 of the first embodiment is described. In the ultrasound diagnosis apparatus 100 of the first embodiment described above, the timing of the intermittent imaging is measured based on a specific ECG waveform or abnormal waveform. However, the first embodiment is not so limited. For example, together with the monitoring by ultrasound examination, the monitoring of subject's heart sound may be performed in parallel using a heart sound monitor. The heart sound monitor includes a phonocardiograph, an analyzer, and the like. The phonocardiograph detects heart sounds by a microphone or a body-conducted sound sensor, converts the sounds to electrical signals, and records them as waveforms. In this structure, the main control unit 104 receives an abnormality detection trigger and waveform data based on various types of extra heart sounds, murmurs, or the like caused, for example, due to heart disease from the heart sound monitor as the biological information measuring unit 120. The main control unit 104 also receives the time interval between sound II and sound I, and an abnormality detection trigger based on a large change in the time interval between sound II and sound I or the like. Information that the heart sound monitor outputs based on the periodic motion of the predetermined site is an example of the “periodic information”. In this manner, upon receipt of an abnormality detection trigger, abnormal waveform, or the like from the heart sound monitor, the main control unit 104 sends a trigger signal to the transmitter/receiver unit 105.

The transmitter/receiver unit 105 controls the end part 10 to acquire as many ultrasound images as corresponding to the heart rate estimated from the heart sound set in advance. Incidentally, the modification 1 can be implemented in combination with the above embodiment using an ECG.

### <Modification 2>

In the following, a modification 2 of the first embodiment is described. In the ultrasound diagnosis apparatus 100 of the first embodiment described above, the timing of the intermittent imaging is measured based on a specific ECG waveform or abnormal waveform. However, the first embodiment is not so limited. For example, together with the monitoring by ultrasound examination, the monitoring of subject's breathing may be performed in parallel using a respiration monitor. The respiration monitor detects the motion of the subject induced by breathing, and outputs a respiration monitor signal. The respiration monitor may be, for example, a pressure sensor band strap that can be placed around the stomach. For another example, the respiration monitor may be an air flow sensor that measures the flow rate of the breathing of the subject. For still another example, the respiration monitor may be a device that captures images of the observation site of the subject using a camera, and analyzes the motion of the observation site in a captured moving image or the like, thereby obtaining the state of motion in the external form of the observation site due to the breathing of the subject.

The respiration monitor generates respiration waveform according to the respiration monitor signal based on the breathing of the subject. The respiration waveform represents a respiration level where the vertical axis indicates time, while the horizontal axis indicates the depth of breathing. For example, in this waveform, the upper direction of the vertical axis indicates high level inhalation, and the lower direction indicates high level exhalation. The intermediate value between the maximum level inhalation and the maximum level exhalation is a boundary value at which the inhalation and the exhalation are switched.

In this structure, the main control unit 104 receives various types of information related to breathing from the respiration monitor as the biological information measuring unit 120. For example, the main control unit 104 receives an abnormality detection trigger based on, for example, abnormal respiratory rate (apnea (including respiratory arrest), slow breathing rate, tachypnea), abnormal ventilation, periodic breathing abnormality (Cheyne-Stokes respiration), irregular abnormality or the like. Information that the respiration monitor outputs based on the periodic motion of the predetermined site is an example of the “periodic information”. In this manner, upon receipt of an abnormality detection trigger or the like, the main control unit 104 sends a trigger signal to the transmitter/receiver unit 105.

The transmitter/receiver unit 105 controls the end part 10 to acquire as many ultrasound images as corresponding to a plurality of cycles of breathing set in advance. Incidentally, the modification 2 can be implemented in combination with both or either one of the modification 1 and the above embodiment using an ECG.

### <Modification 3>

Next, a modification 3 of the first embodiment is described. In this modification, the transmitter/receiver unit 105 of the main body 101 implements the most functions of the transmitter 141 and the receiver 142 of the end part 10. With this, the internal structure of the container 10a may be simplified. Described below is an example of the functions of the transmitter/receiver unit 105.

### (Transmitter Unit—Modification 3)

The transmitter unit of the transmitter/receiver unit 105 of the main body 101 includes a clock generation circuit, a transmitter delay circuit, and a pulser circuit (not illustrated), and the like, which are controlled by the main control unit 104. The clock generation circuit generates clock signals for determining the transmission frequency and the transmission timing of ultrasound waves. For example, the clock generation circuit feeds the transmitter delay circuit with a reference clock signal. The transmitter delay circuit sends the pulser circuit a drive signal having a predetermined delay time. The predetermined delay time is determined based on the transmission focal point of ultrasound waves. The pulser circuit includes therein as many pulsers as individual channels corresponding to the ultrasound oscillators 12a, and generates transmission drive pulses.

The pulser circuit repeatedly generates a rate pulse to form transmission ultrasound waves at a predetermined repetition frequency (PRF). The transmitter delay circuit provides the rate pulse with a delay time related to the transmission direction and the transmission focus. Transmission drive pulses are generated at timing based on the rate pulses each being delayed. The transmission drive pulses are sent to the end part 10 through the cable 11, and fed to the ultrasound oscillators 12a of the ultrasound transducer 12 via the transmit-receive controller 14. The transmission drive pulses excite the piezoelectric elements. As described above, the transmitter delay circuit provides the pulser circuit with a delay time to focus ultrasound waves for transmission, thereby converging the ultrasound waves into a beam. With this, the transmission directivity of the ultrasound waves is determined. In addition, the transmitter delay circuit changes the transmission delay time to be given to each rate pulse, thereby controlling the transmission direction of ultrasound waves from the ultrasound wavefront.

### (Receiver Unit—Modification 3)

The receiver unit of the transmitter/receiver unit 105 of the main body 101 receives echo signals corresponding to ultrasound waves reflected from the subject under the control of the main control unit 104. Having received the echo signals received by the end part 10, the receiver unit performs delay addition processing on them, thereby converting the analog echo signals to digital data having been subjected to phasing (i.e., subjected to beam forming). Specific examples are as follows.

The receiver unit of the transmitter/receiver unit 105 includes, for example, a preamplifier circuit, an A/D converter, a receiver delay circuit, and an adder (all not illustrated). The preamplifier circuit amplifies echo signals received from the ultrasound transducer 12 with respect to each receiver channel. The A/D converter converts the amplified echo signals to digital signals. Having been converted into digital signals, the echo signals are each stored in a digital memory. The digital memory is provided for each channel (or each element). Each echo signal is stored in the corresponding digital memory. The echo signal is also stored in an address corresponding to the time it is received.

The receiver delay circuit provides echo signals converted to digital signals with a delay time required to determine the reception directivity. The reception delay time is calculated for each element. The adder adds up the echo signals having the delay time. The adder reads each of the echo signals from the digital memory as appropriate based on the required delay time calculated, and adds up them. The adder repeats this addition while changing a reception focus position along the transmission beam. The addition emphasizes a reflection component from a direction corresponding to the reception directivity. The received beam signal processed by the transmitter/receiver unit 105 is sent to the signal processor (the B-mode signal processing unit 107, the Doppler signal processing unit 108).

### Second Embodiment

In the following, the ultrasound diagnosis apparatus 100 according to the second embodiment is described. In the first embodiment, information as a trigger to perform the intermittent imaging is received from a device (ECG) that directly detects biological signals of the subject. In the second embodiment, the information as a trigger to perform the intermittent imaging is received from the end part 10. Further, differently from the first embodiment, after receipt of the information that triggers the intermittent imaging, filtering is applied to the information to classify it into a plurality of pieces. The ultrasound diagnosis apparatus 100 of the second embodiment is similar to that of the first embodiment in other respects. Only the differences are described below.

The breathing, pulsation, and the like of the subject induce vibration in the subject's body. The ultrasound diagnosis apparatus 100 of the second embodiment detects the vibration induced in the subject's body by a vibration sensor (not illustrated) provided in the end part 10. For example, along with breathing, pulsation, and the like, vibration occurs in other body tissues (esophagus, etc.). The vibration sensor detects the vibration. In the second embodiment, the vibration sensor repeats the detection process.

### <<Vibration Sensor>>

As the vibration sensor, a three-axis acceleration sensor may be used. For example, if the container 10a is formed in a capsule, small one is preferable. To trigger the intermittent imaging, resolution is also required. From this point of view, a three-axis acceleration sensor may be used. The three-axis acceleration sensor detects signal information about vibration (vibration information) with respect to each of the three axes X, Y, and Z.

### <<End Part>>

The end part 10 includes, in addition to the same ultrasound beam transmitter/receiver system as of the first embodiment, the vibration sensor and a processor that performs processing (amplification, A/D conversion, etc.) on a detection signal detected by the vibration sensor. To transmit the detection signal to the main body 101, a signal line used for transmitting/receiving ultrasound waves through the I/F 15 may be used. A signal line other than the ultrasound system used for the transmission of the detection signal may also be used.

### <<Filtering>>

The main control unit 104 performs filtering on the detection signal received by the main body 101 via the cable 11. Specifically, the main control unit 104 removes noise from the detection signal by filtering. The main control unit 104 extracts component derived from the vibration of body tissues based on heart sound (heart sound information), and component derived from the vibration of body tissues based on the respiratory sound (respiratory information) from the detection signal from which noise is removed by filtering.

### <<Heart Sound>>

The main control unit 104 obtains waveform data based on the data of the detection signal on heart sound extracted. From the waveform, the main control unit 104 obtains the time interval between sound II and sound I, and an abnormality detection trigger based on a large change in the time interval between sound II and sound I or the like. In response to the abnormality detection trigger or abnormal waveform obtained from the detection signal, the main control unit 104 sends a trigger signal to the transmitter/receiver unit 105. The transmitter/receiver unit 105 controls the end part 10 to acquire as many ultrasound images as corresponding to the heart rate estimated from the heart sound set in advance.

### <<Respiration>>

The main control unit 104 obtains waveform data based on the data of the detection signal on heart sound extracted. From the waveform, the main control unit 104 obtains the motion of the subject due to the breathing. The main control unit 104 receives an abnormality detection trigger based on, for example, abnormal respiratory rate (apnea (including respiratory arrest), slow breathing rate, tachypnea), abnormal ventilation, periodic breathing abnormality (Cheyne-Stokes respiration), irregular abnormality or the like. In response to the abnormality detection trigger or the like, the main control unit 104 sends a trigger signal to the transmitter/receiver unit 105.

The transmitter/receiver unit 105 controls the end part 10 to acquire as many ultrasound images as corresponding to a plurality of cycles of breathing set in advance.

In the ultrasound diagnosis apparatus 100 of this embodiment, when the operator provides operation to start monitoring body tissues, the main control unit 104 does not immediately issue a trigger signal, and also the transmitter/receiver unit 105 does not send the end part 10 a signal related to the driving of the ultrasound transducer 12. In this embodiment, the imaging of body tissue is initiated when the main control unit 104 receives a detection signal from the vibration sensor of the end part 10, and obtains an abnormality detection trigger. Further, after the start of the transmission of ultrasound waves, upon completion of the imaging set in advance, the transmitter/receiver unit 105 of the main body 101 terminates the intermittent imaging. With this structure, ultrasound waves can be prevented from being transmitted continuously and all the time in the subject's body. Accordingly, it is possible to avoid heat generation due to the prolonged transmission of ultrasound waves.

Moreover, the ultrasound diagnosis apparatus 100 is configured to acquire ultrasound images while looking for the right time to capture images, such as when there is a change in the conditions of the subject informed from the biological information measuring unit 120. That is, the ultrasound diagnostic apparatus 100 does not continue to acquire ultrasound images if the conditions of the subject are stable for a long period of time. As a result, the viewer of ultrasound images in the monitoring is not forced to examine unnecessary images. Thus, it is possible to reduce the burden on the viewer. This results in the improved efficiency of the ultrasound examination.

As in the first embodiment, the ultrasound diagnosis apparatus 100 of the second embodiment may have the end part 10 in a capsule form. Moreover, the end part 10 may have a structure in which only minimal lines such as a signal line and a power supply line are passed through the cable 11. This makes it possible to reduce the burden on the subject compared to the case of using the TEE probe.

### Third Embodiment

In the following, the third embodiment is described. In the first and the second embodiments, upon acquisition of specific information from information based on a biological signal and a periodic change in the information based on the biological signal, the main control unit 104 sends a trigger signal to the transmitter/receiver unit 105. On the other hand, in the ultrasound diagnosis apparatus 100 of the third embodiment, the main control unit 104 periodically sends a trigger signal related to the transmission of ultrasound waves to the transmitter/receiver unit 105 based on periodic information received from the biological information measuring unit 120. The ultrasound diagnosis apparatus 100 of the third embodiment is similar to that of the first embodiment in other respects. Only the differences are described below.

The main control unit 104 of the third embodiment receives information of characteristic waveform that is periodically produced among waveforms based on biological signals from the biological information measuring unit 120. In the following, an example is described below in which the biological information measuring unit 120 is ECG. In this example, each time the biological information measuring unit 120 indicates P wave, Q wave, R wave, S wave, T wave, and the like in ECG waveforms, the main control unit 104 issues a trigger signal.

### <Operation>

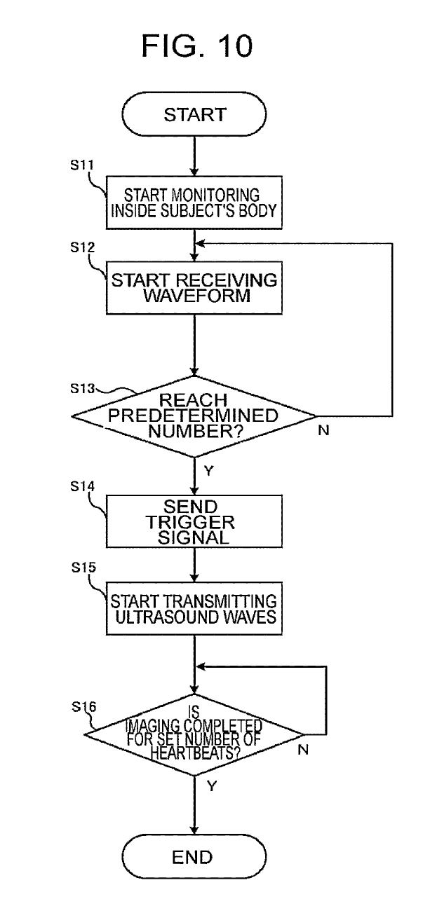

The main control unit 104 controls each unit of ultrasound diagnosis apparatus 100 to generate ultrasound images (including Doppler spectrum images, color flow mapping images, etc.) in the predetermined cardiac time phase. In the following, a description is given of a control flow to perform the intermittent imaging according to the embodiment with reference to FIG. 10. FIG. 10 is a flowchart schematically illustrating the operation of the ultrasound diagnosis apparatus 100 of the third embodiment.

### (Step S11)

When the operator has determined the initial setting, the monitoring of body tissue is started. The initial setting includes, in addition to the items described in the first embodiment, when the heart is to be monitored, setting of desired cardiac time phase in which the operator wants to generate ultrasound images. The following explanation is made as to the monitoring of the heart where setting is determined in advance such that Doppler spectrum images are acquired in diastole. As for the heart rate as a reference to intermittently capture ultrasound images, setting is determined such that images are captured for 2 heartbeats of every 20 heartbeats.

### (Step S12)

Having started measuring ECG waveform, the biological information measuring unit 120 sends a trigger signal to the main control unit 104 at the timing when a specific waveform is indicated to enable the acquisition of ultrasound images in the predetermined cardiac time phase, i.e., in diastole. For example, at the timing when R wave and T wave are indicated in ECG waveform acquired in real time, the biological information measuring unit 120 sends a trigger signal to the main control unit 104.

### (Step S13)

From the start of the monitoring in step S01, having received a trigger signal based on a specific waveform (R wave, etc.) from the biological information measuring unit 120, the main control unit 104 obtains the heart rate of the subject based on the trigger signal. In the third embodiment, a predetermined number of heartbeats is set in the initial setting as the interval at which the intermittent imaging is performed. The main control unit 104 determines whether the heart rate of the subject reaches the predetermined number. In the example of step S11, the predetermined number is set at 20 heartbeats. Accordingly, the main control unit counts the heart rate of the subject based on the trigger signal from the start of the monitoring and repeats steps S12 and S13 (step S13; No) until counting 20 heartbeats.

### (Step S14)

In step S13, having determined that the heart rate of the subject reaches the predetermined number, i.e., 20 heartbeats (Step S13; Yes), the main control unit 104 receives, for example, a trigger signal corresponding to R wave and that corresponding to T wave from the biological information measuring unit 120. The main control unit 104 sends a trigger signal to the transmitter/receiver unit 105 to acquire ultrasound images in the predetermined cardiac time phase, i.e., Doppler spectrum images in the pulsed Doppler mode in diastole.

### (Step S15)

Upon receipt of the trigger signal, the transmitter/receiver unit 105 reads data of the number of heartbeats set in advance for the intermittent imaging from the storage unit (not illustrated). Having retrieved the data of the number of heartbeats, the transmitter/receiver unit 105 makes the end part 10 transmit/receive ultrasound waves based on the trigger signal corresponding to R wave and that corresponding to T wave received from the main control unit 104.

### (Step S16)

When the end part 10 starts transmitting/receiving ultrasound waves in step S15, the receiver unit receives an echo signal, and the generating unit 109 generates an ultrasound image through several types of signal processing. Having started receiving ECG waveforms as well as transmitting ultrasound waves, the transmitter/receiver unit 105 starts measuring the timing to terminate the intermittent imaging from these time points. That is, the transmitter/receiver unit 105 determines whether the imaging is completed for the number of heartbeats set in advance based on, for example, ECG waveform received in real time. When the imaging has not yet been completed (Step S16; No), the transmitter/receiver unit 105 repeats this determination.

For another example, the main control unit 104 may be configured to obtain the time taken for one heartbeat from ECG waveforms being received in real time. That is, the main control unit 104 obtains the imaging time for a predetermined number of heartbeats based on the time of one heartbeat. Having determined that the imaging for the predetermined number of heartbeats is completed, the main control unit 104 sends a trigger indicating the end of the imaging to the transmitter/receiver unit 105.

When determining that the intermittent imaging is completed (step S16; Yes), the transmitter/receiver unit 105 terminates the intermittent imaging without transmitting a signal related to the driving of the ultrasound transducer 12.

In the ultrasound diagnosis apparatus 100 of this embodiment, when the operator provides operation to start monitoring body tissues, the main control unit 104 does not immediately issue a trigger signal, and also the transmitter/receiver unit 105 does not send the end part 10 a signal related to the driving of the ultrasound transducer 12. In this embodiment, the imaging is performed in response to the regular transmission of a trigger signal related to the transmission of ultrasound waves to the transmitter/receiver unit 105 based on periodic information received from the biological information measuring unit 120. Further, after the start of the transmission of ultrasound waves, upon completion of the imaging set in advance, the transmitter/receiver unit 105 of the main body 101 terminates the intermittent imaging. With this structure, ultrasound waves can be prevented from being transmitted continuously and all the time in the subject's body. Accordingly, it is possible to avoid heat generation due to the prolonged transmission of ultrasound waves.

Moreover, the ultrasound diagnosis apparatus 100 is configured to acquire ultrasound images while looking for the right time to capture images, such as when there is a change in the conditions of the subject informed from the biological information measuring unit 120. That is, the ultrasound diagnostic apparatus 100 does not continue to acquire ultrasound images if the conditions of the subject are stable for a long period of time. As a result, the viewer of ultrasound images in the monitoring is not forced to examine unnecessary images. Thus, it is possible to reduce the burden on the viewer. This results in the improved efficiency of the ultrasound examination.

As in the first embodiment, the ultrasound diagnosis apparatus 100 of the third embodiment may have the end part 10 in a capsule form. Moreover, the end part 10 may have a structure in which only minimal lines such as a signal line and a power supply line are passed through the cable 11. This makes it possible to reduce the burden on the subject compared to the case of using the TEE probe.

### Forth Embodiment

In the following, the forth embodiment is described. In the third embodiment, the main control unit 104 periodically sends a trigger signal related to the transmission of ultrasound waves to the transmitter/receiver unit 105 based on periodic information received from the biological information measuring unit 120. On the other hand, in the ultrasound diagnosis apparatus 100 of the forth embodiment, the main control unit 104 obtains the time of one heartbeat of the subject, and performs the intermittent imaging based on the time and the time interval set in advance at which images are captured. The ultrasound diagnosis apparatus 100 of the forth embodiment is similar to that of the third embodiment in other respects. Only the differences are described below.

In the fourth embodiment, when the operator has determined the initial setting, the monitoring of body tissue is started. The initial setting includes, in addition to the items described in the first embodiment, setting of the time interval at which images are captured. For example, the time interval as a reference upon intermittently capturing images is set at 20 seconds. In the initial setting, a time period of the imaging is also set. With these settings, in the fourth embodiment, for example, it is set that images are captured in 2 seconds of every 20 seconds. The set time period is stored in the storage unit (not illustrated).

The main control unit 104 determines whether the set time period has elapsed from the start of monitoring. When the time period has elapsed, the main control unit 104 sends a trigger signal to start the transmission of ultrasound waves to the transmitter/receiver unit 105. Having received the trigger signal, the transmitter/receiver unit 105 controls the end part 10 to start transmitting ultrasound waves based on the initial setting. The transmitter/receiver unit 105 also reads the data of the set imaging time, and determines whether the imaging time has elapsed from the start of ultrasound beam transmission.

When the imaging time has elapsed, the transmitter/receiver unit 105 controls the end part 10 to stop transmitting ultrasound waves. After the end of the imaging, the main control unit 104 counts the time until the next imaging. In the fourth embodiment, even if the ultrasound diagnosis apparatus 100 is not provided with the function of generating a trigger signal based on a biological signal, and even if the ultrasound diagnosis apparatus 100 is not connected to a device having the function of generating a trigger signal, the intermittent imaging can be performed.

In the fourth embodiment, the trigger signal corresponds to time period information related to the elapse of a plurality of cycles set according to the periodic motion of a predetermined site, and is in response to the elapse of the first time period in the time period information. In addition, the trigger signal is capable of making the end part 10 transmit ultrasound waves during the second time period which is less than the first time period. The time period information may be obtained from heartbeats, pulsation, or heart sound based on the motion of the predetermined site.

### Fifth Embodiment

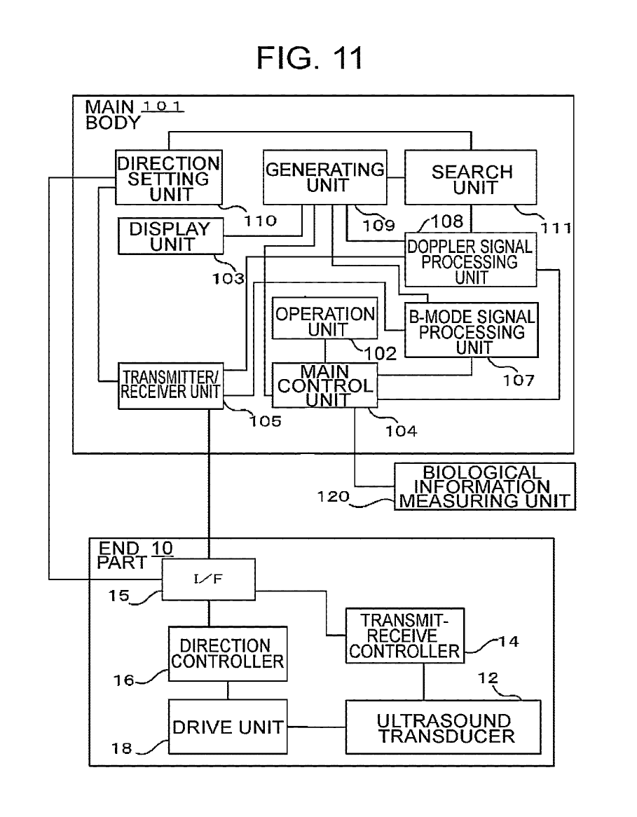

In the following, the fifth embodiment is described with reference to FIGS. 11 to 14. FIG. 11 is a schematic block diagram illustrating an example of the functional structure of a main body of an ultrasound diagnosis apparatus according to the fifth embodiment. As illustrated in FIG. 11, the main body 101 of the fifth embodiment includes the search unit 111.

### (Direction Setting Unit)

The direction setting unit 110 of the embodiment receives transmission direction data from the search unit 111, and sets the transmission direction as well as performing the function described in the first embodiment. The details are described in the explanation of the search unit 111.

Incidentally, the direction setting unit 110 corresponds to an example of a “changer”. Besides, in combination with the direction controller 16 and the drive unit 18 of the end part 10, the direction setting unit 110 corresponds to an example of a “changer”. In combination with the transmitter/receiver unit 105 and the transmit-receive controller 14 of the end part 10, the direction setting unit 110 corresponds to an example of a “changer”.

### (Search Unit)

While the ultrasound diagnosis apparatus 100 is transmitting/receiving ultrasound waves to obtain an ultrasound image, for adjusting the transmission direction of the ultrasound waves and the position of an area to be examined, the search unit 111 searches for the transmission direction of the ultrasound waves. The search is based on a Doppler signal obtained by transmitting/receiving ultrasound waves in the Doppler mode. Specifically, the search is performed by determining whether the transmission direction of ultrasound waves in the Doppler signal (or sample volume) adjusts to the desired observation object that produces a blood flow. When the adjustment is performed by the search unit 111 as a precondition, if any scan mode is selected by the operator, the main control unit 104 controls the end part 10 to acquire the Doppler signal in parallel with the acquisition of the ultrasound image. Note that the Doppler signal indicates an echo signal obtained in the Doppler mode, or the RAW data of the Doppler image having subjected to the signal processing by the signal processor. For convenience of the description, the Doppler signal may be similarly described below. In addition, the Doppler mode indicates any one of scan modes for obtaining blood flow information, including pulsed Doppler mode, continuous wave Doppler mode, color Doppler mode, power Doppler mode, and the like. For convenience of the description, the Doppler mode may be similarly described below.

For example, while the B-mode is selected and a B-mode image is generated, the main control unit 104 prompts the operator to set sample volume on the B-mode image BI displayed. When sample volume is set by the operator, according to a control signal received from the transmitter/receiver unit 105, the end part 10 alternately repeats B-mode scanning and the acquisition of Doppler signals in the pulsed Doppler mode. Based on the Doppler signals acquired, the search unit 111 performs the search process for the adjustment of the transmission direction of ultrasound waves and the position of an area to be examined. For example, the search unit 111 can be used to search for the transmission direction of ultrasound waves in the ultrasound transducer 12 upon monitoring cardiac ejection fraction.

Comparing pieces of signal strength information indicating the strength of Doppler signals obtained over time, the search unit 111 of the first mode determines the transmission direction of ultrasound waves with the highest signal strength. Described below is an example of the search process performed by the search unit 111.

### <<Start of Transmission of Ultrasound Waves>>

After the end part 10 is inserted in the subject's body and scan mode is selected by the operator for preparation, the transmission of ultrasound waves is started. The receiver unit of the transmitter/receiver unit 105 of the main body 101 acquires echo signals based on the scan mode over time. The signal processor, the generating unit 109, and the like generate ultrasound images corresponding to the scan mode based on the echo signals. The display unit 103 displays the ultrasound images as appropriate. If the Doppler mode is selected as scan mode, only echo signals based on the selected scan mode are obtained. In other words, switching of the scan mode is not performed.

### <<Start of Search>>

If the scan mode is the B mode, the B-mode signal processing unit 107 sends RAW data based on the echo signals to the generating unit 109. The Doppler signal processing unit 108 sends Doppler signals to the search unit 111. The transmitter/receiver unit 105 of the main body 101 starts transmitting ultrasound waves in the Doppler mode for the search process of the search unit 111. Triggered by the elapse of a predetermined time (any time that is set) from the start of the transmission, the transmitter/receiver unit 105 makes the end part 10 transmit ultrasound waves in the Doppler mode. At this time, the direction setting unit 110 controls the end part 10 so that it transmits ultrasound waves not only in the direction in which ultrasound waves are transmitted first, but transmits ultrasound waves while changing the transmission direction. The time interval at which the search process is performed can be set arbitrarily.

### <<Ultrasound Transmission Based on ECG Waveform>>

In the search process, ultrasound waves can be transmitted while the transmission direction is changed at any time interval set by the operator. For example, based on ECG waveform received from the biological information measuring unit 120, the main control unit 104 obtains predetermined cardiac time phase (diastole phase, etc.). The main control unit 104 may send the transmitter/receiver unit 105 a control signal related to the transmission timing of ultrasound waves with respect to each cardiac time phase thus obtained. The predetermined cardiac time phase refers to diastole or systole, early systole, mid-systole, end-systole, early diastole, mid-diastole, end-diastole or the like. Note that, in the search process, the main control unit 104 is not necessarily configured to transmit a control signal related to the transmission timing of ultrasound waves in the predetermined cardiac time phase. For another example, the main control unit 104 may be configured to obtain the predetermined cardiac time phase from ECG waveform received from the biological information measuring unit 120, and determine the signal strength (described later) of a Doppler signal corresponding to the predetermined cardiac time phase among Doppler signals acquired successively.

Also when the search unit 111 performs the search process, the initial setting of the Doppler mode is required. For example, the main control unit 104 notifies the operator of the start of selected scan mode, or prompts the operator to set sample volume before or after it. As the notification, for example, a predetermined character string may be displayed on the display unit 103, or voice guidance may be output. After a predetermined time has elapsed, first, the direction setting unit 110 makes the end part 10 transmit ultrasound waves via the transmitter/receiver unit 105 in the transmission direction corresponding to the initial setting. Then, the direction setting unit 110 makes the end part 10 transmit ultrasound waves via the transmitter/receiver unit 105 towards around the transmission direction of the initial setting, for example, in directions adjacent to the transmission direction of the initial setting.

### <<Acquisition of Signal Strength Information>>

In the Doppler mode, the receiver unit of the transmitter/receiver unit 105 sequentially obtains Doppler signals transmitted in different directions. The Doppler signals are those obtained by the Doppler signal processing unit 108, and derived from blood flow (if the observation object is blood flow: blood flow PWD or CWD), or derived from tissues (if the observation object is tissues: tissue PWD). In the following, unless otherwise noted, the observation object is described as blood flow. In this case, it is assumed that a signal derived from blood flow, from which components derived from tissues that represent noise are removed, is extracted as a Doppler signal. The Doppler signal processing unit 108 sends a Doppler signal to the search unit 111. Together with information on the transmission direction of ultrasound waves, the search unit 111 stores Doppler signals obtained sequentially from the signal processor in a storage unit (not illustrated). From each of the stored Doppler signals transmitted in different directions, the search unit 111 acquires signal strength information that indicates the strength of the signal. The signal strength information is, for example, blood flow sensitivity information in the pulsed Doppler mode. In this case, the blood flow sensitivity information may be the amplitude value or the brightness value of a waveform depicted in a Doppler spectrum image. Each time the search unit 111 obtains a Doppler signal, the search unit 111 may acquire signal strength information from the Doppler signal. In this case, the search unit 111 stores, in the storage unit (not illustrated), signal strength information obtained sequentially and information on the transmission direction of ultrasound waves.

### <<Comparison of Signal Strength>>

Besides, the search unit 111 compares Doppler signals in different directions corresponding to, for example, predetermined cardiac time phase, and obtains a Doppler signal having a higher signal strength. By the comparison of signal strength, a Doppler signal indicating the highest signal strength is stored together with information on the corresponding transmission direction of ultrasound waves. The search unit 111 may acquire the signal strength upon acquisition of each Doppler signal. The search unit 111 may also be configured to obtain the highest signal strength from Doppler signals at each time point, after the completion of the search process described below.

### <<End of Search>>

The transmission of ultrasound waves and the process of acquiring Doppler signals corresponding thereto continue, under the control of the direction setting unit 110, until a predetermined condition is satisfied. Examples of the predetermined condition include completion of predetermined times of transmission, completion of transmission in a predetermined range (a predetermined angle range from the sound source), elapse of a predetermined time, and the like. Upon receipt of a Doppler signal obtained last in a cycle, the search unit 111 obtains signal strength information determining that it is the end of the cycle. Then, the search unit 111 compares Doppler signals with a Doppler signal having the highest signal strength in an earlier cycle. With this comparison, the search unit 111 completes one cycle of the search process, and determines information on the transmission direction of ultrasound waves corresponding to a Doppler signal having the highest signal strength. The search unit 111 transmits the information on the transmission direction of ultrasound waves thus determined to the direction setting unit 110.

### <<Update of Direction Setting>>

The direction setting unit 110 compares the information on the transmission direction of ultrasound waves received from the search unit 111 to the transmission direction of ultrasound waves before the search process. If there is a difference between them, based on the information on the transmission direction of ultrasound waves received from the search unit 111, the direction setting unit 110 updates the setting of the transmission direction of ultrasound waves. In addition, based on the updated setting, the direction setting unit 110 changes the transmission direction of ultrasound waves to a new direction through the transmitter 141 of the end part 10, or the direction controller 16 and the drive unit 18. The direction setting unit 110 and the search unit 111 of this embodiment correspond to an example of a “controller”.

The above is an example of the search process by the search unit 111. As an another example, when the continuous wave Doppler mode is initially selected by the operator, the signal strength of a Doppler signal may be obtained in response to the start of the transmission of ultrasound waves without waiting for the elapse of a predetermined time as described above. In this case, changes in signal strength in the same transmission direction may be continuously obtained based on Doppler signals acquired sequentially. However, in the continuous wave Doppler mode, ultrasound waves are continuously transmitted and received. Therefore, it is preferable to search for the transmission direction of ultrasound waves as well as changing the transmission direction also at predetermined time intervals, in the same manner as the search of the transmission direction based on the signal strength as described above.

Due to the breathing, beats, body movement, throat reflection, emetic response, and the like of the subject, the transmission direction of ultrasound waves sometimes shifts from the object observed by the ultrasound diagnosis apparatus. In particular, if the observation object shifts not in the depth direction in the transmission direction of ultrasound waves, but in a direction deviating from the direction (orthogonal direction, etc.), it is difficult to continue the monitoring by the ultrasound diagnosis apparatus. Thus, each time a shift occurs, it is required to adjust the rotation and tilt of the ultrasound transducer 12 of the end part 10, the focus and transmission direction of ultrasound beams, and the like. Alternatively, each time a shift occurs, it is required to adjust the sample volume location (depth).

PWD mode has a range resolution. For example, during monitoring in the PWD mode, as well as the adjustment of the transmission direction of ultrasound beams, the sample volume location (depth) is adjusted with respect to the distance direction in the sound ray (scan line) of the ultrasound beams.

On the other hand, CWD mode has no range resolution. For example, during monitoring in the CWD mode, adjustment is performed for obtaining a location (depth) where the signal strength of a Doppler signal is the highest while the focus position (depth) of ultrasound beams is being changed.

However, it may be a heavy burden for the operator to keep monitoring shifts and also adjust them. If the operator bears these tasks, it may cause a decrease in the efficiency of monitoring inside the subject's body by the ultrasound diagnosis apparatus. In the case of long-term monitoring, since it is difficult for the operator to keep monitoring whether the transmission direction of ultrasound waves is appropriate, it may interfere with the implementation of the monitoring. In this respect, the ultrasound diagnosis apparatus 100 includes the search unit 111 as described above to periodically adjust the transmission direction of ultrasound waves, thus solving the problems. That is, it is possible to improve the operation efficiency without imposing burdensome tasks on the operator in monitoring inside the subject's body. Moreover, the ultrasound diagnosis apparatus 100 can effectively cope with long-term monitoring.

### <Operation>

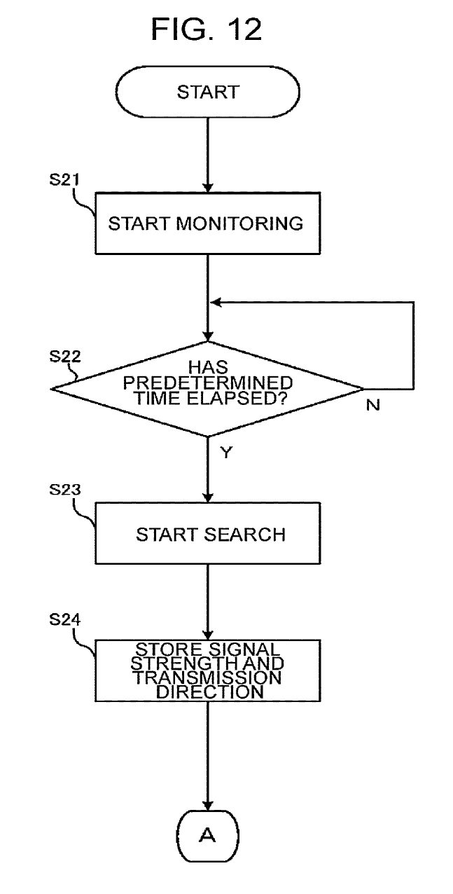

In the following, a description is given of a control flow to perform the search process at predetermined time intervals as well as displaying a B-mode image, a Doppler spectrum image, and ECG waveform in parallel according to the embodiment with reference to FIGS. 12 to 14. FIGS. 12 to 14 are flowcharts schematically illustrating the operation of the ultrasound diagnosis apparatus 100 of the fifth embodiment.

### (Step S21)

When the operator determines the initial setting using the operation unit 102, the main control unit 104 controls the intermittent imaging as in the above embodiments.

### (Step S22)

The main control unit 104 determines whether a predetermined time has elapsed from the start of the monitoring. If, in step S22, determining that the predetermined time (e.g., any time period set by the operator) has not elapsed (Step S22; No), the main control unit 104 repeats this determination.

### (Step S23)

In step S22, having determined that the predetermined time has elapsed (step S22; Yes), the main control unit 104 makes the end part 10 start transmitting/receiving ultrasound waves via the transmitter/receiver unit 105 for the search process. If a B-mode image BI is displayed on the display unit 103 (see FIG. 6), the main control unit 104 may prompt the operator to specify the position of sample volume. The operator specifies any region of the B-mode image BI as sample volume on the operation unit 102. In FIG. 6, the transmission direction is indicated by a broken line L1 extending from the left atrium LA through the mitral valve M to the left chamber, and passing by the center of the left heart. The specified sample volume is sent to the direction setting unit 110, and the direction setting unit 110 sends information on the transmission direction of ultrasound waves from the sound source to the end part 10 via the transmitter/receiver unit 105. The position of the sample volume may be specified prior to step S23.

### (Step S24)

The transmitter/receiver unit 105 receives an echo signal based on the Doppler mode from the end part 10. The Doppler signal processing unit 108 performs the signal processing on the echo signal to obtain a Doppler signal and sends the Doppler signal to the search unit 111. The search unit 111 generates signal strength information based on the Doppler signal corresponding to predetermined cardiac time phase. The signal strength information generated by the search unit 111 is stored in the storage unit (not illustrated) with the information on the transmission direction of ultrasound waves.

### (Step S25)

Based on the ECG waveform fed from the biological information measuring unit 120, the main control unit 104 measures the timing of the next transmission of ultrasound waves in the search process. The main control unit 104 repeats this (step S25; No) until the timing of the next transmission of ultrasound waves.

### (Step S26)

In step S25, having determined that it is the timing of the next transmission of ultrasound waves based on the ECG waveform (step S25; Yes), the main control unit 104 controls the direction setting unit 110 so that the end part 10 transmits ultrasound waves after changing the transmission direction of ultrasound waves from the direction initially set to a direction around it. If the scan mode in the initial setting is not the Doppler mode, at the arrival of the time for transmitting ultrasound waves, the main control unit 104 changes the transmission direction of ultrasound waves by the direction setting unit 110 after switching the scan mode to the Doppler mode.

### (Step S27)

Having received an echo signal related to ultrasound waves transmitted by changing the transmission direction, the receiver unit of the transmitter/receiver unit 105 sends the signal to the Doppler signal processing unit 108. The search unit 111 generates signal strength information based on the Doppler signal received from the Doppler signal processing unit 108, and stores it in the storage unit (not illustrated) with the information on the corresponding transmission direction of ultrasound waves. The main control unit 104 obtains the predetermined cardiac time phase based on the ECG waveform fed from the biological information measuring unit 120. The main control unit 104 also obtains signal strength corresponding to the predetermined cardiac time phase from among Doppler signals acquired successively.

### (Step S28)

The main control unit 104 determines whether the termination condition of the search process, such as completion of predetermined times of transmission, completion of transmission in a predetermined range (a predetermined angle range from the sound source), elapse of a predetermined time, and the like, is satisfied. In step S28, having determined that the termination condition is not satisfied (step S28; No), the main control unit 104 repeats steps S25 to S28.

### (Step S29)

In step S28, if the main control unit 104 determines that the termination condition of the search process is satisfied (step S28; Yes), the search unit 111 retrieves pieces of the signal strength information from the storage unit (not illustrated) and compares them. The search unit 111 may be configured to compare signal strength information with prior one each time signal strength information is obtained through the repetition of steps S25 to S27. In this case, since the provisional highest signal strength has already been determined, the search unit 111 compares signal strength obtained most recently with the provisional highest signal strength at the previous time.

### (Step S30)

According to the result of the comparison in step S29, the search unit 111 determines the transmission direction of ultrasound waves with the highest signal strength.

### (Step S31)

The search unit 111 sends the direction setting unit 110 information on the transmission direction of ultrasound waves thus determined.

### (Step S32)

Comparing the transmission direction set in advance with the information on the transmission direction received in step S31, the direction setting unit 110 determines whether there is a difference between them.

### (Step S33)

In step S32, having determined that there is a difference (step S32; Yes), the direction setting unit 110 updates the setting of the transmission direction of ultrasound waves based on the information on the transmission direction of ultrasound waves received in step S31.

### (Step S34)

The direction setting unit 110 determines whether the ultrasound transducer 12 is required to be rotated or tilted by the direction controller 16 and the drive unit 18 based on the updated setting.

### (Step S35)

In step S34, having determined that the ultrasound transducer 12 is required to be rotated or tilted (step S34; Yes), the direction setting unit 110 rotates or tilts the ultrasound transducer 12 with the direction controller 16 and the drive unit 18. However, when the 2D array ultrasound transducer 12 is used, there may be a case where this determination is not necessary.

### (Step S36)

The direction setting unit 110 changes the transmission direction of ultrasound waves to a new direction for the monitoring by the intermittent imaging. In step S34, having determined that the ultrasound transducer 12 is not required to be rotated or tilted (step S34; No), the direction setting unit 110 performs this without performing step S35.

In step S32, having determined that there is no difference (step S32; No), the direction setting unit 110 ends the process without performing steps S33 to S36.

### <Modification 1>

Described below is a modification 1 of the fifth embodiment. The ultrasound diagnosis apparatus 100 of the fifth embodiment is configured to search for the optimal transmission direction of ultrasound waves based on signal strength obtained by the search process. However, the fifth embodiment is not so limited. For example, the search unit 111 may perform the search process based on a waveform indicating blood flow information generated by the generating unit 109.

### <<Generation of Reference Waveform Data>>

The second waveform data as a reference is stored in the storage unit (not illustrated). The second waveform is compared with the first waveform that is generated sequentially in the search process. The second waveform data is generated in advance, for example, at the start of monitoring, or before or after it. The second waveform data corresponds to predetermined cardiac time phase.

### <<Start of Search>>

The transmitter/receiver unit 105 of the main body 101 starts transmitting ultrasound waves in the Doppler mode to obtain the first waveform used in the search process of the search unit 111. Triggered by the elapse of a predetermined time from when the second waveform is acquired, the transmitter/receiver unit 105 makes the end part 10 transmit ultrasound waves in the Doppler mode. The time interval at which the search process is performed can be set arbitrarily.

### <<Ultrasound Transmission Based on ECG Waveform>>

In the search process, the time interval, at which ultrasound waves are transmitted while the transmission direction is changed, is set correspondingly to cardiac time phase in the second waveform.

### <<Generation of Waveform Image>>

The Doppler signal processing unit 108 performs the same signal processing as in the fifth embodiment on echo signals received from the transmitter/receiver unit 105, thereby sending RAW data of Doppler spectrum images to the generating unit 109. The generating unit 109 sequentially generates Doppler spectrum images based on the RAW data. The waveform may be based on M-mode images (images collected in M mode), provided that the first waveform and the second waveform are obtained in the same scan mode.

### <<Generation of First Waveform>>

At this time, the main control unit 104 obtains cardiac time phase corresponding to that of the second waveform from the ECG waveform fed by the biological information measuring unit 120, and send it to the search unit 111. The search unit 111 extracts a waveform in the cardiac time phase corresponding to that in the second waveform from the waveform image generated by the generating unit 109. The search unit 111 uses this waveform as the first waveform.

### <<Calculation of Similarity of Waveforms>>

The search unit 111 determines the similarity between the second waveform stored and each of the first waveforms generated sequentially in the search process. The similarity can be obtained by, for example, cross-correlation operation. When the overlapping area of the first waveform and the second waveform is at the peak, the search unit 112 determines it as representing high similarity, and obtains phase difference between the two at this time. The search unit 111 determines the similarity of the two waveforms based on the phase difference. The similarity information thus obtained is stored in the storage unit (not illustrated) with information on the transmission direction of ultrasound waves.

### <<Comparison of Similarity>>

Comparing the first waveforms in different directions, the search unit 111 determines one which is more similar to the second waveform. The first waveform with the highest similarity in the comparison is stored with information on the corresponding transmission direction of ultrasound waves.

In this modification, the optimal transmission direction of ultrasound waves is searched for as described above. Regarding the transmission direction information, the direction setting unit 110 operates in the same manner as in the fifth embodiment. The modification 1 can be combined with the fifth embodiment.

The ultrasound diagnosis apparatus 100 of this embodiment transmits ultrasound waves at predetermined intervals in the transmission direction set in advance and directions around it, and acquires a plurality of Doppler signals corresponding to different transmission directions. The search unit 111 searches for the optimal transmission direction of ultrasound waves based on the Doppler signals. If a position shift has occurred, the direction setting unit 110 changes the transmission direction of ultrasound waves to the optimal transmission direction. Thus, even if the end part 10 shifts in the subject's body due to the breathing, beats, body movement, throat reflection, emetic response, and the like of the subject, and the transmission direction of ultrasound waves shifts from the object to be observed, it is possible to change the transmission direction of ultrasound waves to follow the shift, thereby enabling the continuation of monitoring inside the subject's body without imposing burdensome tasks on the operator. Moreover, even in long-term monitoring, it is possible to avoid a decrease in the operation efficiency.

### Sixth Embodiment

In the following, the sixth embodiment is described. The ultrasound diagnosis apparatus 100 of the fifth embodiment is configured to search for the optimal transmission direction of ultrasound wave through the search process by the search unit 111. The sixth embodiment is the same in this respect. However, the search unit of the sixth embodiment further performs, when there is found no suitable transmission direction of ultrasound waves, error notification, termination of ultrasound beam monitoring (transmitting/receiving ultrasound waves), and the like. Otherwise, the ultrasound diagnosis apparatus 100 of the sixth embodiment is similar to that of the fifth embodiment. Only the differences are described below.

### (Search Process—Signal Strength)

The search unit 111 of the sixth embodiment stores a threshold for signal strength. In the search process, having determined the highest signal strength, the search unit 111 compares the signal strength with the threshold. If the signal strength is below the threshold, the search unit 111 determines that there is found no suitable transmission direction of ultrasound waves. Then, the search unit 111 notifies, via a notification unit (not illustrated), the operator of recognizable error information. For example, the notification unit displays an error message on the display unit 103. For another example, the notification unit outputs predetermined sound from an audio output unit (not illustrated). In this case, the search unit 111 does not send information on the transmission direction of ultrasound waves to the direction setting unit 110.

As another operation of the search unit 111, if the signal strength is below the threshold, the search unit 111 determines that there is found no suitable transmission direction of ultrasound waves. Then, the search unit 111 informs the main control unit 104 of this. Upon receipt of the information, the main control unit 104 stops the transmission of ultrasound waves by the end part 10. As an example of the situation where the search unit 111 cannot find the suitable transmission direction of ultrasound waves may be cited a case where the shift of the end part 10 is large. In this case, the observation object is likely to be not included in ROI even if the direction setting unit 110 rotates/tilts the ultrasound transducer 12 and changes the transmission direction of ultrasound waves by electronic scanning.

### (Search Process—Similarity)

The search unit 111 of the sixth embodiment stores a threshold for similarity. In the search process, having determined the transmission direction of ultrasound waves with the highest similarity, the search unit 111 compares the similarity with the threshold. If the similarity is below the threshold, the search unit 111 determines that there is found no suitable transmission direction of ultrasound waves. Then, the search unit 111 notifies, via the notification unit (not illustrated), the operator of recognizable error information. The notification unit operates in the same manner as described above. Also, the main control unit 104 stops the transmission of ultrasound waves by the end part 10 in the same manner as described above.

In this embodiment, the ultrasound diagnosis apparatus 100 is configured to perform, when there is found no optimal transmission direction of ultrasound waves, error notification, termination of ultrasound beam transmission, and the like. For example, if the observation object is not included in ROI even by rotating/tilting the ultrasound transducer 12 and changing the transmission direction of ultrasound waves in electronic scanning, the operator needs to recognize the situation. In addition, the end part 10 is required to be removed in such a situation. In this respect, according to the embodiment, even when the end part 10 has shifted largely with respect to the subject, the operator can handle the situation appropriately.

With the ultrasound diagnosis apparatus 100 according to the first to sixth embodiments described above, the imaging is performed intermittently according to the periodic motion or conditions of body tissues of the subject. With this structure, ultrasound waves can be prevented from being transmitted continuously and all the time in the subject's body. Thus, it is possible to avoid heat generation due to the prolonged transmission of ultrasound waves.

The first to the sixth embodiments can be used in any combination as appropriate. In the embodiments, not only the end part 10 in a capsule form, but a TEE probe may also be used.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus, comprising:
an ultrasound transducer configured to transmit and receive ultrasound waves while being inserted in a subject to acquire biological information of a predetermined site of the subject; and
a controller configured to control the ultrasound transducer to transmit ultrasound waves based on a trigger signal that is set according to conditions of the predetermined site that exhibits periodic motion, or that is obtained according to the conditions.

2. The ultrasound diagnosis apparatus according to claim 1, wherein the trigger signal is obtained based on non-periodic motion of the predetermined site.

3. The ultrasound diagnosis apparatus according to claim 2, wherein the controller is configured to receive, from a biological information measuring unit that is configured to continuously monitor the periodic motion or the non-periodic motion of the predetermined site, the trigger signal based on the motion.

4. The ultrasound diagnosis apparatus according to claim 3, wherein
the biological information measuring unit is an electrocardiograph, and
the controller is configured to receive the trigger signal based on the non-periodic motion of the predetermined site from the electrocardiograph.

5. The ultrasound diagnosis apparatus according to claim 4, wherein
the electrocardiograph is configured to perform analysis of electrocardiographic waveform, and
the controller is configured to receive the trigger signal output as a result of the analysis in response to occurrence of non-periodic electrocardiographic waveform.

6. The ultrasound diagnosis apparatus according to claim 3, wherein
the biological information measuring unit is a heart sound monitor or a respiration monitor, and
the controller is configured to receive the trigger signal based on the non-periodic motion from the heart sound monitor or the respiration monitor.

7. The ultrasound diagnosis apparatus according to claim 6, wherein the controller is configured to receive the trigger signal and periodic information based on the periodic motion from the heart sound monitor or the respiration monitor, and, upon receipt of the trigger signal, control the ultrasound transducer to transmit ultrasound waves over a plurality of cycles based on the periodic information.

8. The ultrasound diagnosis apparatus according to claim 2, further comprising:
a container configured to house the ultrasound transducer; and
a vibration sensor housed in the container, and configured to detect vibration based on the periodic motion or the non-periodic motion of the predetermined site.

9. The ultrasound diagnosis apparatus according to claim 8, wherein
the trigger signal includes vibration information related to the vibration detected by the vibration sensor, and
the controller is configured to
perform processing on the trigger signal, and classify the vibration information into heart sound information based on heart sound of the subject and respiration information based on breathing of the subject,
detect non-periodic heart sound from the heart sound information and control the ultrasound transducer to transmit ultrasound waves, and
detect non-periodic breathing from the respiration information and control the ultrasound transducer to transmit ultrasound waves.

10. The ultrasound diagnosis apparatus according to claim 3, wherein
the biological information measuring unit is an electrocardiograph, and
the controller is configured to
receive the trigger signal from the electrocardiograph, and
obtain cardiac time phase, which is set in advance, based on the trigger signal, and control the ultrasound transducer to transmit ultrasound waves according to the cardiac time phase.

11. The ultrasound diagnosis apparatus according to claim 2, wherein
the trigger signal is time period information that is related to elapse of a plurality of cycles and set according to the periodic motion of the predetermined site, and
the controller is configured to, in response to elapse of first time period in the time period information, control the ultrasound transducer to transmit ultrasound waves for second time period that is shorter than the first time period.

12. The ultrasound diagnosis apparatus according to claim 11, wherein the time period information is obtained from heartbeats, pulsation, or heart sound based on the motion of the predetermined site

13. The ultrasound diagnosis apparatus according to claim 3, wherein
the biological information measuring unit is an electrocardiograph, and
the controller is configured to count number of heartbeats based on a signal from the electrocardiograph, and receive the trigger signal when the number of heartbeats reaches a predetermined value.

14. The ultrasound diagnosis apparatus according to claim 1, wherein
the ultrasound transducer includes a changer configured to change transmission direction of ultrasound waves, and is configured to transmit ultrasound waves in a direction set while being inserted in the subject; and
the controller is configured to determine a direction toward the predetermined site based on the biological information, and control the changer to adjust the transmission direction of ultrasound waves to the direction.

15. The ultrasound diagnosis apparatus according to claim 1, further comprising:
a capsule container configured to house at least the ultrasound transducer;
a main body;
an interface configured to transmit and receive signals between the ultrasound transducer and the main body; and
a power supply line configured to supply power at least to the ultrasound transducer.

16. The ultrasound diagnosis apparatus according to claim wherein the container is in a capsule form, and includes:
the ultrasound transducer;
an interface configured to transmit and receive signals between the ultrasound transducer and a main body as an external unit with respect to the container; and
a power supply line configured to supply power at least to the ultrasound transducer.

17. The ultrasound diagnosis apparatus according to claim 15, wherein
the main body includes:
a power supply connected to the power supply line; and
a signal processor connected to the controller and the interface, and configured to perform processing on signals based on reflected waves received from the ultrasound transducer, and
the container is configured to be connected to the main body by the interface.
